SEQUENCE LISTING (2 sequences)

SEQ ID NO: 1 - protein, 41 aa, Homo sapiens
SYSTEMANDMETHODFORPREDICTINGMICROPROTEINS

SEQ ID NO: 2 - protein, 35 aa, synthetic construct
HSLQRGTFKTQNTRSRLQLRDSEAKLEGLRKDEEY

(19) United States
(12) Patent Application Publication
(10) Pub. No.: US 2025/0201349 A1
(43) Pub. Date: Jun. 19, 2025

(54) SYSTEM AND METHOD FOR PREDICTING MICROPROTEINS

(71) Applicant: Research Development Foundation, Carson City, NV (US)

(72) Inventors: Brendan Miller, La Jolla, CA (US); Eduardo Vieira de Souza, La Jolla, CA (US); Alan Saghatelian, La Jolla, CA (US)

(21) Appl. No.: 18/975,313

(22) Filed: Dec. 10, 2024

Related U.S. Application Data
(60) Provisional application No. 63/638,075, filed on Apr. 24, 2024.
(60) Provisional application No. 63/608,642, filed on Dec. 11, 2023.

(51) Int. Cl. G16B 40/20; G16B 30/00

(52) U.S. Cl. CPC G16B 40/20; G16B 30/00

(57) ABSTRACT

Techniques for training and using a machine learning model to perform microprotein prediction. One computer-implemented method includes, accessing a set of data describing expressed amino acid sequences, and generating decoy training data that includes randomized amino acid sequences that are matched to properties of the amino acid sequences included in the set of data. The method then includes training a machine learning model using labeled training data and the decoy training data, the labeled training data including amino acid sequences within a set of classifications and the decoy training data constituting examples of amino acid sequences that are not expected to be within the set of classifications. The trained machine learning model is usable to receive an input that describes a structure of a particular amino acid sequence, and perform a classification of the particular amino acid sequence relative to the set of classifications.

The present application claims priority to U.S. Provisional App. No. 63/638,075, entitled “NEURAL NETWORK SYSTEMS FOR smORF PEPTIDES,” filed Apr. 24, 2024, and U.S. Provisional App. No. 63/608,642, entitled “NEURAL NETWORK SYSTEMS FOR smORF PEPTIDES,” filed Dec. 11, 2023; the disclosures of each of the above-referenced applications are incorporated by reference herein in their entireties.

### INCORPORATION OF SEQUENCE LISTING

The sequence listing that is contained in the file named “OP0024-US-0850 Sequence Listing.xml”, which is 25,788 bytes (as measured in Microsoft Windows) and was created on Dec. 27, 2024, is filed herewith by electronic submission and is incorporated by reference herein.

### BACKGROUND

One of the most fundamental questions in all of biology is the number of proteins encoded within an organism's genome, and this knowledge was a driving force behind genome sequencing projects. The completion of Human Genome Project in 2003, for example, was supposed to identify all protein coding genes once and for all; however, intense debates regarding the count of protein-coding genes are ongoing because of the recent discovery of tens of thousands of unannotated encoded peptides and small proteins, collectively referred to as microproteins, in the human genome. Microproteins are short sequences of amino acids that are encoded by small open reading frames (smORFs) (e.g., 150 or fewer amino acids, 100 or fewer amino acids). Until recently, microproteins were unannotated, which we define as being absent from any primary gene/protein databases, but a community effort that aimed to define guidelines for the inclusion of microproteins recently added about 7,500 microprotein-encoding smORFs to GENCODE, a database that is regularly updated to contain all annotated human proteins. These smORFs were annotated across multiple different cell types with similar methodologies. Should these smORFs encode microproteins, it would expand the number of known human proteins (i.e. the human proteome) by nearly 50%.

The recent discovery of microproteins comes from advances in proteomics and genomics for detecting unannotated protein products and the translation of open reading frames (ORFs), respectively. The aforementioned 7,500 smORFs, for example, emerged from ribosome profiling, or Ribo-Seq, experiments, a widely adopted genomics technology. Ribo-Seq entails the chemical inhibition of RNA translation within live cells (e.g., using cycloheximide) to stall the ribosome onto RNAs, though in some formats this step is excluded. Subsequently, the ribosome-RNA complexes, referred to as polysomes, is isolated, then nuclease digested to generate ribosome protected fragments (RPFs) that are then sequenced, and aligned to a reference transcriptome to identify sites of active RNA translation, including actively translated smORFs. Precise codon-level alignment enables accurate identification of actively translated smORFs from RNA fragments that are protected by other RNA-binding proteins. Besides the ˜7,500 smORFs noted in GENCODE, numerous other Ribo-Seq experiments have been conducted, leading to the inference of tens of thousands previously unannotated smORFs in human as well as other organisms.

The biology of smORF-encoded microproteins is nuanced, as active translation doesn't guarantee that the microproteins are stable or functional. Many microproteins lack significant evolutionary conservation (i.e., conversation between species such as humans and mice), suggesting that are not functional or emerged during evolution after speciation. Notably, about 90% of GENCODE's annotated smORFs are ‘evolutionarily young,’ with roughly three-quarters displaying conserved microprotein structures across primatomorpha. But relying solely on evolutionary conservation may prove insufficient for specific microprotein candidate validation. Some of these recently evolved microproteins interact physically with essential proteins. smORFs in the 5′- and 3′-UTRs have also been shown to be functional in translation so in some cases the functional element might be the smORF and not the microprotein. For instance, the ATF4 mRNA transcript contains an smORF in its 5′-UTR, referred to as an upstream ORF (uORF), that regulates ORF translation between the uORF (the smORF) and downstream ORF during cellular stress. However, there have been tens of microproteins that regulate biochemistry, cell biology, or physiology, which are referred to as positively annotated or negatively annotated microproteins, that demonstrate that microproteins have biological activities. A corollary to this is that as some microproteins could be involved in disease biology, and it is reasonable to expect that some of these microproteins will be eventually be drug targets in the future. Thus, it remains crucial to conduct experiments post-Ribo-Seq to validate microprotein existence and roles in biology.

Recent research has found factors that influence the emergence of de novo proteins. One study highlighted the importance of the hydrophobic carboxy terminus (C-terminus) in directing evolutionarily novel translated peptides to the BAG6 machinery, potentially leading them to the proteasome or cellular organelle membranes (29). Interestingly, this study unveiled an evolutionary trend favoring less hydrophobic C-termini, establishing a correlation between a gene's age and C-terminal hydrophobicity in both humans and mice. This implies that, even when specific sequences are not conserved, certain protein characteristics experience selective pressure. In a separate investigation, a comparison between a library of de novo proteins and a random protein library underscored the slightly heightened solubility of de novo proteins. This trait is further influenced by the DnaK chaperone system (30). Therefore, while using a strategy based solely on evolutionary conservation is powerful, a limitation in solely relying on these tools is that they might miss recently evolved new proteins (31). Therefore, complementary approaches that seamlessly integrate with existing methods for microprotein discovery are needed.

To highlight the functional capacity of newly discovered microproteins we provide several examples. The microprotein ELABELA is a hormone consisting of a mere 54-amino acids. It was unveiled as a vital player in human embryonic stem cell survival, acting by binding to the Apelin receptor. Similarly, the 54-amino-acid intracellular microprotein PIGBOS regulates the unfolded protein response within the endoplasmic reticulum. Another noteworthy microprotein, TINCR, spanning 120-amino acids, serves as a tumor suppressor in squamous cell carcinoma. Furthermore, several microproteins have emerged as pivotal regulators of mitochondrial function, including BRAWNIN (71-amino acids, encoded by a long non-coding RNA), MIEF1 (70-amino acids, encoded by an upstream open reading frame), Mitoregulin (76-amino acids, encoded by a long non-coding RNA), and SHMOOSE (58-amino acids, encoded by polycistronic mitochondrial RNA). The biology of these microproteins, many of which discovered initially through Ribo-Seq, highlights the functional potential of microproteins but also makes the point that only a small fraction of all microproteins (<1%) have been functionally tested, highlighting the need for methods to rapidly annotate smORFs and microproteins from biologically relevant samples.

Ribo-Seq is an in vitro technique that can be used to detect mRNA expression within a cell or tissue based on the location and abundance of ribosomes in a cell; however, Ribo-Seq has several significant limitations. Ribo-Seq experiments are typically rather expensive and slow. The time required to perform Ribo-Seq analysis, including library preparation, sequencing, and data analysis, can vary depending on the complexity of the experiment and the sequencing platform used, but typically takes several days to a couple of weeks; with the majority of the time spent on sequencing and downstream data analysis. Data from Ribo-Seq can be inconsistent and require multiple studies and analyses, and technical limitations can preclude the use of small biological samples obtained during clinical studies. Some of these challenges can be addressed by running replicates but this increases cost and time, and still does not fully solve the issues with the Ribo-Seq callers. Additionally, RPFs (ribosome protected fragments) that are sequenced span 27-35 base pairs (nucleotides). At these lengths, some RPFs are removed from the Ribo-Seq dataset because they cannot be mapped to a specific site, these RPFs are referred to as multi-mappers. Because of this Ribo-Seq also misses smORFs and microproteins in from multi-mapper sites—i.e., false negatives. Furthermore, smORFs are context-dependent in their translation. For example, during cellular stress responses, eIF2α inactivation can alter smORF translation, and the dynamic regulation of mRNA structures can affect ribosome occupancy on smORFs. Consequently, a single Ribo-Seq experiment is susceptible to the conditions where the data was collected and could therefore miss smORFs and microproteins. These challenges underscore the need for additional methods to identify microprotein-encoding smORFs that can effectively overcome these issues to complement or replace Ribo-Seq in microprotein discovery pipelines.

Another approach for microprotein discovery is to search proteomics data with an in silico 3-frame translation of the transcriptome to identify non-annotated proteins, and this approach is commonly referred to as proteogenomics. Notable microproteins discovered via proteogenomics include NBDY (UniProt: A0A0U1RRE5) and CYREN (UniProt: Q9BWK5). However, microproteins contain fewer tryptic sites than canonical proteins, are potentially less abundant, and might not ionize well, all of which lead to false negatives. For example, we identified the TINCR microprotein (UniProt: A0A2R8Y7D0) by Ribo-Seq, but not by proteogenomics, but subsequent studies readily identified this microprotein using anti-TINCR antibodies in a Western blot. Thus, proteogenomics has even more false negatives than Ribo-Seq, again pointing to the need for improved methods for microprotein discovery.

### BRIEF DESCRIPTION OF THE DRAWINGS

FIG. 1 is a block diagram showing one embodiment of training a machine learning model in accordance with the present disclosure;

FIG. 2A-C is a diagram illustrating evaluation metrics of a neural net trained in accordance with an embodiment hereof, where-FIG. 2A shows 3 components of Uniform Manifold Approximation and Projection (UMAP) for dimension reduction,

FIG. 2B illustrates 3 distinct patterns of visual representation of UMAP,

FIG. 2C shows assigning a class to the GENCODE smORF set;

FIG. 3 is three graphs, A, B, C illustrating the evaluation metrics of a one embodiment of a process in accordance with the present disclosure;

FIG. 4 is five graphs, A-E, showing the prediction of microproteins using the trained neural network;

FIG. 5 is a diagram, A, B, C, illustrating a biological validation of a predicted microprotein for the StAR transcript using a trained neural network;

FIG. 6 is a flow chart showing steps for training a machine learning model and predicting microproteins using the trained machine learning model.

FIGS. 7A-C illustrates an example showing the true positive and negative microprotein set for training purposes where—

FIG. 7(A) is a plot and graph of the true positives sourced from UniProt with cellular compartments annotated from GO;

FIG. 7 (B) is a plot and graph showing true positive secreted or intracellular and negative short proteins using UMAP following feature reduction;

FIG. 7 (C) is a plot and table illustrating a label spreading algorithm treating the GENCODE Ribo-Seq smORFs as unknowns, and using the known true secreted, intracellular, and negative classes to assign and update labels;

FIGS. 8A-F illustrates an example of the system and method hereof applied to a deeply sequenced de novo human transcriptome, where—

FIG. 8(A) is a Venn diagram of the shared number of predicted functionally active microproteins between HEK293T and K562 cells;

FIG. 8 (B) is a Pie graph of the types of smORFs encoding ShortStop predicted functionally active microproteins;

FIG. 8 (C) is a chart illustrating the number of predicted functionally active microproteins by cell type and with RNA counts>10, RNA counts>100, and ShortStop score>90%;

FIG. 8 (D) is a graph illustrating the cumulative fraction of predicted microproteins as a function of RNA count, log-scaled. E.g., nearly half of predicted microproteins have RNA counts greater than 100;

FIG. 8 (E) is a graph illustrating the cumulative fraction of predicted microproteins as a function of ShortStop score (prediction percentage for intracellular or secreted positive subclasses);

FIG. 8 (F) is a graph of a predicted microprotein encoded by a smORF on the KATB transcript aligned across multiple species;

FIGS. 9A-D broadly illustrate peptide-level detection of predicted microproteins in HEK293T and K562 cell lysates where—

FIG. 9(A) is a Venn Diagram of the predicted active microproteins in MS experiments of low molecular weight enriched HEK293T and K562 cell lysates;

FIG. 9 (B) is a Pie graph of the types of smORFs with peptide-level detection in HEK293T and K562 cells lysates;

FIG. 9 (C-D) are graphs showing detection of unique tryptic peptides (MS/MS spectrum shown) for two Shortstop predicted microproteins;

FIGS. 10A-E illustrates the feature characteristics of higher confident predictions and subclasses predictions where—

FIG. 10(A) is a violin plot representing the distribution of microproteins according to each max PhyloCSF score (y axis), which is also color-coded by max PhyloCSF;

FIG. 10(B) is a chart showing C-terminal hydrophobicity of predicted microproteins;

FIG. 10(C) is a bar chart illustrating prediction percentage histogram of predicted active microproteins;

FIG. 10(D) is a graph of the length of predicted microproteins;

FIG. 10(E) is a table showing residue frequency per microprotein for the intracellular and secreted predicted classes;

FIGS. 11A-E relate to the RNA expression of predicted microproteins and immunopeptidomic detection in lung cancer where—

FIG. 11(A) is a diagram and plot where Bulk RNA-Seq re-analysis of lung cancer in never smokers (LCINS) from the Sherlock-Lung study;

FIG. 11 (B) is a hierarchical clustering plot of the first 500 most significantly differentially expressed microprotein smORFs between tumors and normal tissue samples;

FIG. 11 (C) is a diagram and plot where immunopeptidomics re-analysis of two pairs of lung cancer samples with matched normal tissues of HLA class-1 bound peptides;

FIG. 11 (D) is a box plot representation of TXNDC17-MP in normal vs. tumor samples;

FIG. 11 (E) is a bar chart of MS2 of TXNDC17-MP detected as tumor HLA1 immunopeptides, with the detected fragment color-coded;

FIGS. 12A-D illustrates a highly specific antiserum raised in rabbit using a synthetic peptide fragment of StAR-MP where—

FIG. 12(A) is a table showing immunofluorescence staining of HEK293T cells transiently transfected with a StAR-MP construct, visualized with the anti-StAR-MP serum followed by a goat anti-rabbit IgG highly cross-absorbed secondary antibody Alexa488. Cells were counterstained with DAPI to visualize transfected and control transfected cells;

FIG. 12 (B) top is a western immunoblot of StAR-MP from HEK293T cell lysates transiently transfected with control construct and bottom blot indicates total protein transferred as evident by Coomassie staining; and

FIG. 12 (C-D) are graphs showing displacement of [125I-Tyr38]-StAR-MP(4-38) peptide binding to rabbit antiserum against StAR-MP by synthetic StAR-MP(4-38) peptide and, from left to right, human testis extracts and human ovary extracts. The Y axis is the B/Bo (%), and the X axis is the total amount of protein in pg.

FIG. 13 is a flow diagram of one embodiment of a method for training a machine learning model for classifying an amino acid sequence.

FIG. 14 is a flow diagram of one embodiment of a method for using a machine learning model to classify an amino acid sequence.

FIG. 15 is a block diagram of one embodiment of a computer system that may be used to implement techniques disclosed herein.

### DETAILED DESCRIPTION

Machine learning's rising appeal in gene discovery makes it tempting to apply these methods to microprotein discovery. Yet, a core hurdle is the absence of a definitive dataset with clear positive and negative microprotein examples. For instance, not all “non-coding RNAs” are truly non-coding (like BRAWNIN, TINCR, DANCR mRNAs). In addition, since some microprotein-encoding smORFs do not produce functional proteins it would also be useful for any machine learning approach to identify microproteins with amino acid sequences that are similar to annotated protein and microproteins (e.g., those with accession numbers in UniProt). We refer to this group as our positive annotation set with assigned Gene Ontological terms and are Swiss-Prot reviewed, which means that they are translated, stable, and detectable, and for some of them have defined functions. We chose this route because including this data would bias our predictions for microproteins that are more likely to be well translated and stable, which is prerequisite for any microprotein that has a biological function. This set might also include de novo microproteins that are translated and stable, but have not evolved to have a biological role yet.

Several machine learning tools have surfaced for smORF prediction, and these all rely heavily on genomics data but no protein data. Moreover, the lack of a well-defined set of true positives and negatives restricts their utility and comparative evaluation. Faced with this conundrum, two options emerge. The first option involves developing new molecular and biochemical tools to enhance confidence in defining positive and negative training sets. But this approach is resource-intensive and time-intensive. Undoubtedly, though, the field will continue to advance in designing these tools and make progress. Option two focuses on using top-quality data, including gene and protein data, to develop innovative machine learning-based tools for microprotein prediction.

Broadly speaking, the present disclosure contemplates a system and method for predicting microproteins using machine learning techniques. In one embodiment, a machine learning model for predicting microproteins is trained. In one form a system and method hereof leverages Ribo-Seq and traditional protein annotation to create a predictive machine learning model, enabling various downstream applications. For example, one application is to the sequenced human transcriptome to generate a predicted microproteome. These predictions can lead to the discovery of a microprotein encoded by an overlapping upstream smORF on the StAR-encoding transcript. Another example indicates that predicted microproteins enriched in HLA complexes are upregulated in cancer.

In a broad form, a method in accordance with one embodiment trains a machine learning model to predict microproteins using several data sets. For example, a first data set defining proteins with known classes and encodes the first data as a first input. A decoy data input matched to a set of data and encoding the decoy data input as a negative input. The neural network is trained using the first and decoy data inputs. The trained machine learning model may be operated by inputting on or more open reading frames (ORFs), preferably small open reading frames, <about 150 amino acids into the trained machine learning model, and outputting a classification of said input ORF. In some embodiments, the classification is expressed as a probability of inactive, intracellular function, or secreted. In some embodiments, a second data defining proteins with unknown classes and encoding the second data is also used as an input to train the machine learning model.

One exemplary system to model small open reading frames (smORFs) in accordance with one embodiment of the present disclosure broadly predicts the probability a microprotein is likely to be negatively annotated or annotated as intracellular and secreted, the system and includes one or more computer systems having access to one or more non-transitory computer readable media having instructions which, when executed by the one or more computer systems, operates to train a machine learning model. In one embodiment involving semi-supervised learning, the training uses first data defining proteins with known classes and encoding the first data as an input; second data defining proteins with unknown classes and encoding the second data as an input; and generating a decoy data input matched to physical properties of proteins in the second data and encoding the decoy data input as a negative input. The first, second, and decoy data are processed to train the machine learning model to classify which smORF falls into the positive annotation or negative microprotein annotation set. That is, one or more small smORFs are input into the trained machine learning model, and the probability the input translated microprotein is classified as intracellular, secreted, or negative is then produced. In one implementation, the probability output is expressed as negative and secreted versus intracellular.

Genome annotation is the process of identifying functional elements along the sequence of a genome. “Positively annotated” as used herein indicates that a genomic or nucleotide (e.g., a DNA or RNA, preferably a smORF) sequence that has been indicated as or is likely transcribed into a protein (e.g., a microprotein) in a cell (e.g., a mammalian cell or human cell), wherein the protein may have a biological function. A positively annotated genomic sequence (e.g., a smORF) may be further indicated or characterized as producing a protein (e.g., a microprotein) that is expected to likely ether: (i) reside in the intracellular portion of a cell or within the cell membrane (i.e., an “intracellular” positively annotated sequence), or (ii) be secreted by the cell to the extracellular environment (a “secreted” positively annotated sequence). Thus, systems, software, and methods provided herein can be used to further classify positively annotated sequences as predicted to generating proteins that are either intracellular or extracellular. The systems, software, and methods provided herein can be used to identify select genomic sequences that are both positively annotated and wherein the protein (e.g., microprotein) is predicted as residing in the intracellular portion of the cell or be secreted to the extracellular environment outside of the cell. A positively annotated nucleotide sequence may or may not have a specific biological activity within the cell, and in some instances the positively annotated sequence is transcribed into a stable protein that does not have any particular biological function in the cell. Nonetheless, it is anticipated that the methods and systems provided herein can be used in the process of identifying positively annotated sequences that both are transcribed into a protein (e.g., a microprotein) that also has a biological activity (e.g., either inside of the cell or after being secreted by the cell). Preferably, positively annotated sequences are transcribed into a stable protein (e.g., a stable microprotein). “Negatively annotated” as used herein indicates that a genomic or nucleotide (e.g., a DNA or RNA, preferably a smORF) sequence that is indicated or predicted to not be transcribed into a stable protein. Because negatively annotated sequences are not predicted to produce a stable protein, the sequence would also be predicted not to produce a protein with any biological function within the cell.

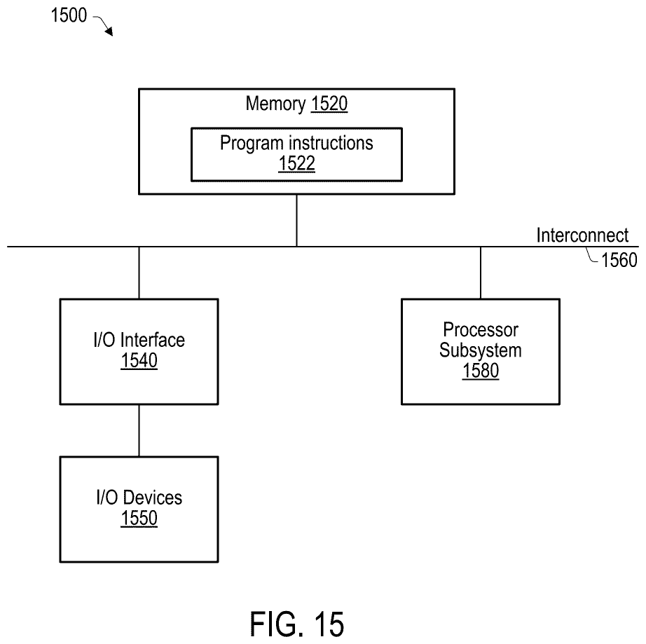

In one implementation, the machine learning model is a neural network. Neural networks are a form of machine learning typically modeled to operate like a biological neural circuit by having a plurality of interconnected neurons or nodes. These nodes can be implemented using a computer system such as that described below with reference to FIG. 15. The nodes can optionally be arranged in a plurality of layers such as input layer, output layer, and one or more hidden layers. Each node is connected to one or more other nodes in the neural network. Each node is configured to receive an input, implement a function and provide an output in accordance with the function. Additionally, each node is associated with a respective weight. Neural networks are trained with one or more data sets to minimize a cost function, which is a measure of the neural network's performance. Typically the training algorithm uses node weights and/or bias to minimize a cost function. There are many algorithms that find the minimum of the cost function and can be used to for training a neural network. There are many types of neural networks, convolutional neural network (CNN) and artificial neural networks (ANNs) being common. Neural networks can operate as function approximation, e.g. prediction and modeling, classification, and data processing.

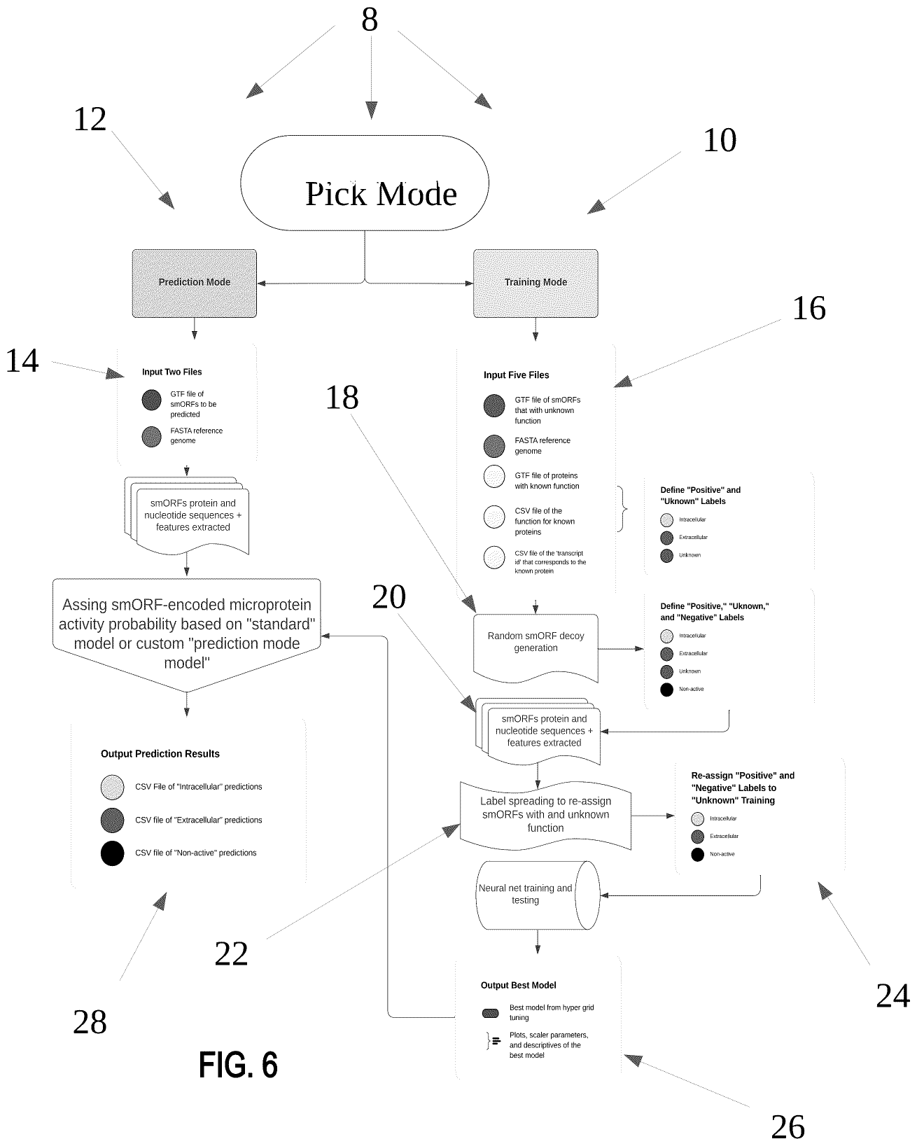

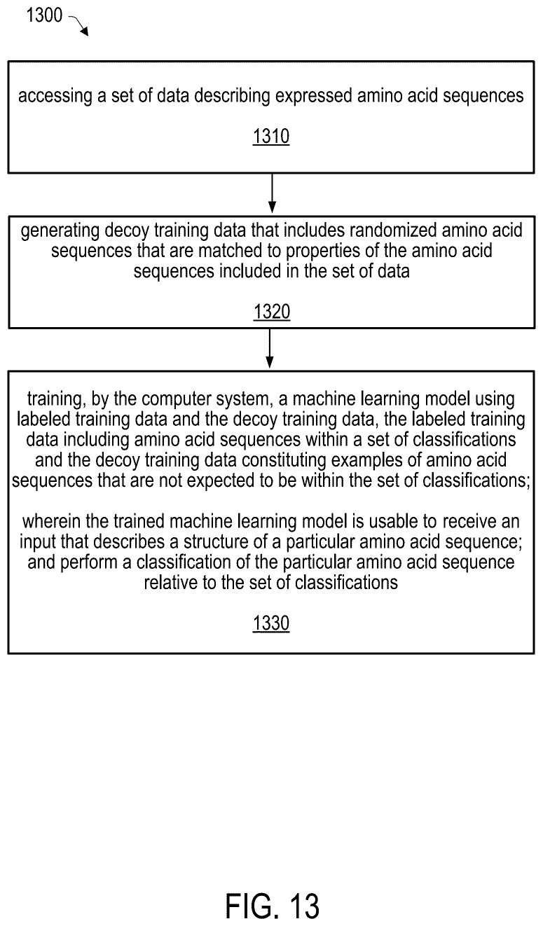

FIG. 6 broadly illustrates a flow one embodiment of a process in accordance with the present disclosure. The right-hand side of FIG. 6, labeled 10 represents a broad view of a flow for “training” the neural network 8. The left-hand side of FIG. 6 at 12 represents how a trained neural network is used to predict the of an input smORF as at 14. In training process 10, a number of training data sets are input as at 16. A random smORF decoy dataset is created at 18. smORFs protein and nucleotide sequences and features are extracted at 20, with label spreading at 22. Semi supervised learning occurs at step 24, with the result being a trained machine learning model as at 26. As described below with reference to FIG. 13, in some embodiments, semi-supervised learning is not used.

The training process, FIG. 6 at 10, may involve creating custom neural nets to predict a user-defined set of protein classes. In training process, the user inputs proteins with known classes (e.g., intracellular, extracellular, etc.) and a list of unknown/unlabeled microproteins (e.g., from Ribo-Seq). Following input of data sets 26, a decoy/negative lass is generated 18, labels assigned to the unlabeled microproteins, and a machine learning model 26 is trained to classify microproteins.

The trained machine learning model 26 takes in a file with genetic coordinates of putative smORFs (i.e., GTF fille) 14 and outputs 28 a classification preferably expressed as the probability of the microprotein is positive (intracellular in function, or extracellular in function). This is generally seen at FIG. 6, 12. The software implementation of FIG. 6 was written in Python and includes nearly two-dozen custom classes that are integrated into a pipeline (e.g., GTFtoSeq class takes a GTF file and outputs the protein and transcript sequences). The software can be used as a shell-based executable tool and in this embodiment uses tensor flow, sikitlearn, and protelearn classes.

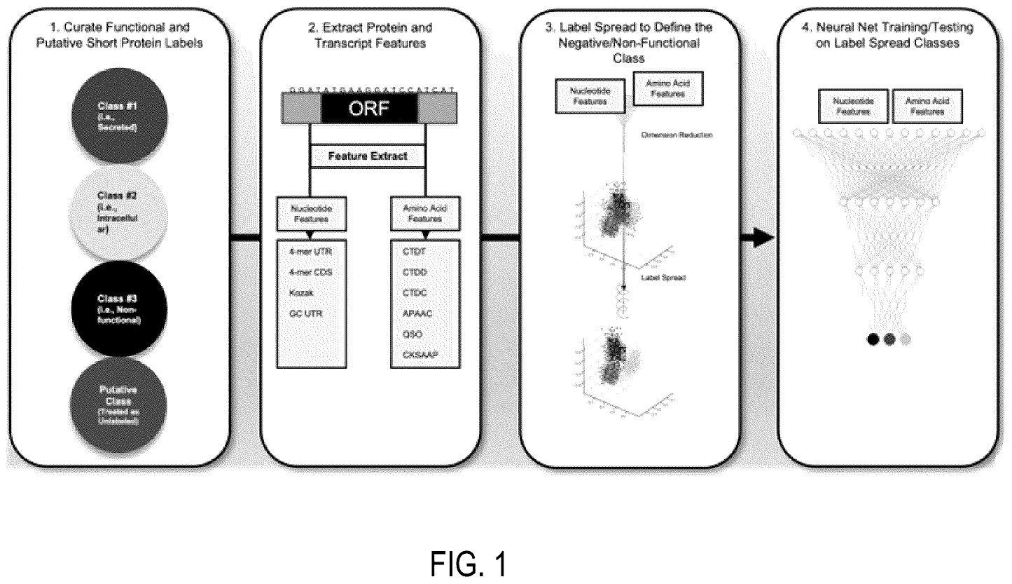

Turning to FIG. 1, the training of a machine learning model in accordance with one embodiment preferably utilizes the ‘Phase 1’ catalog of Ribo-Seq-derived smORFs, known as GENCODE smORFs.’ This GENCODE catalog comprises 7,246 smORFs, each exceeding 16 amino acids, extracted from seven publications that employed Ribo-Seq for genome-wide ORF annotation. Notably, 3,085 smORFs were identified in multiple studies. The key advantage of the GENCODE smORF catalog lies in its consistent methodology across various cell types in the studies.

One embodiment of the present disclosure also uses a training dataset of short proteins from Swiss-Prot, which were deposited in UniProt and associated with Gene Ontology Cellular Compartment terms. This dataset included 1,525 proteins, with 464 having a “secreted” GO term and 1,061 lacking a “secreted” GO term. During training, this embodiment considers these two categories, secreted and intracellular proteins, as “positive” examples.

The microprotein functional annotation status of the 7,246 GENCODE smORFs in encoding microproteins remains uncertain. Some may act as translation regulators, while others may function through the microproteins they encode. In one approach these smORFs are not pre-assigned labels, treating them as microprotein-agnostic during the training process.

Importantly, the theoretical framework of this embodiment relies on a randomly generated decoy ORF set that is treated with a “negative” label. This randomly generated decoy set of ORFs is matched to the distribution of ORF lengths and codon probabilities provided in the ‘unknowns.’ That is, the decoy set is matched to the GENCODE smORFs. As a result, the decoy ORF set establishes a seed for a semi-supervised approach. In this sense, this embodiment establishes a true “negative” set and true “positive” set for training purposes.

Following the decoy ORF generation, each ORF may undergo feature extraction at the nucleotide and amino acid levels as broadly depicted in FIG. 1 at step 2. At the nucleotide level, 4-mers of the 5′ UTR, 3′ UTR, and first 50 CDS were calculated; and GC content of the 5′ UTR, 3′UTR, and first 50 base pairs of the CDS were extracted. At the protein level, features derived from the APAAC, CTD, QSO and CTDD methods were extracted.

After feature extraction, the embodiment of FIG. 1 deploys a label spreading algorithm in reduced dimensional space to assign one of three labels to the GENCODE smORFs (intracellular, secreted, or negative). The embodiment of FIG. 1 uses Uniform Manifold Approximation and Projection for Dimension Reduction (UMAP) as the dimension reduction technique. UMAP uses Riemannian geometry and algebraic topology, enabling it to maintain efficient computation times while also preserving both local and global data structures. To limit bias during training, this embodiment restricts smORFs that encode potential microproteins over 29 amino acids in length, as the shortest protein encoded by the nuclear genome in UniProt is 25 amino acids. A user of the embodiment of FIG. 1 may adjust this parameter during the ‘training’ mode if so desired. The general schematic of this workflow is illustrated in FIG. 1.

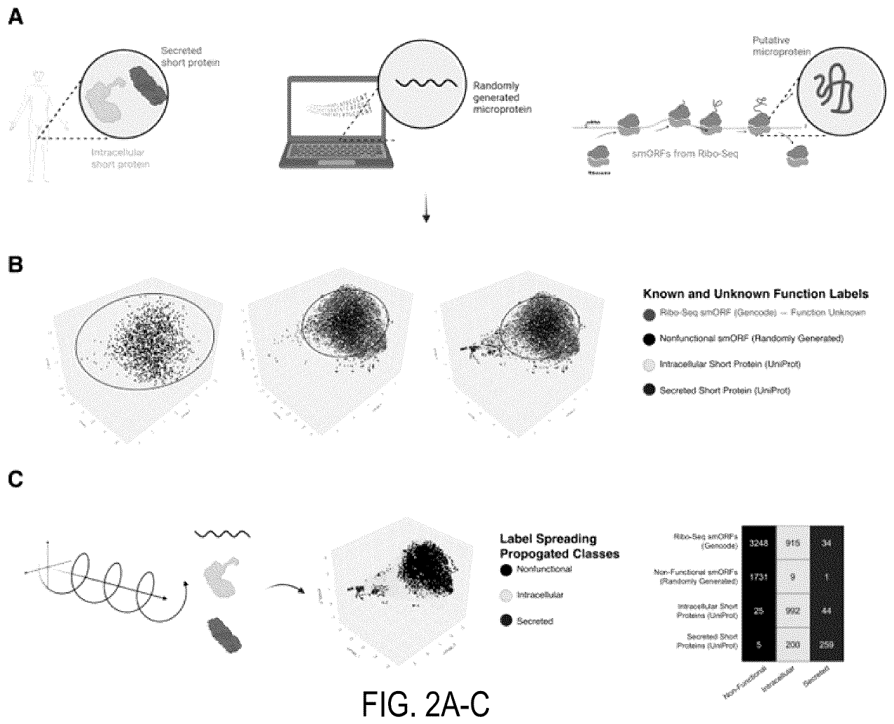

FIG. 2 broadly illustrates one labeling approach. Prior to label spreading, UMAP was carried out on 1,696 features with 100 nearest neighbors and three components (FIG. 2A). The visual representation of UMAP showed three distinct patterns. First, the randomly generated ‘decoy’ set of ORFs appeared close together (black spheres, FIG. 2B) Second, the GENCODE smORFs clustered predominantly with the negative decoy set (red spheres, FIG. 2B), suggesting that a majority of smORFs shared features of the negative annotation set (i.e., the microprotein lacks functional certainty). However, hundreds of smORFs deviated from the focal point of the feature reduction in a manner that clustered with the positive intracellular (yellow spheres) and secreted proteins (blue spheres) translated from ORFs deposited in UniProt (FIG. 2B). This suggests that, while many smORFs cluster with the negative annotation set, a significant number of smORFs appear to cluster with proteins that share features of known, biologically annotated short proteins.

Subsequently, the rbf function is called from a label spreading algorithm for semi-supervised learning. The purpose of this semi-supervised learning is to assign a class to the ‘unknown’ GENCODE smORF set and capture more information for downstream algorithm development. In this example, after label spreading, 915 GENCODE smORFs were labeled as intracellular (21.6%), 34 as secreted (0.9%), and 3,239 as microprotein negative (77.6%). Of the 1748 randomly generated ORFs, just 9 were labeled as intracellular and 1 was labeled as secreted. For the positive ORFs downloaded from UniProt with labels intracellular or secreted, 992 of the original 1,061 intracellular proteins retained their original label; of the secreted class, 259 of the original 464 retained their original label, indicating that representation of intracellular short proteins is more prevalent (more on this in the discussion) (FIG. 2C). These labels were assigned to the GENCODE smORFs during supervised classification training in the following section.

One embodiment develops a machine learning model to classify smORFs into one of three categories derived from label spreading: (1) negative, (2) intracellular, or (3) secreted. In one implementation, the classification is expressed as a probability a category exists. The idea of using a machine learning model is centered on the anticipated downstream application. For instance, a primary use of the methods and systems described herein is large transcriptome-wide data sets that contains millions of putative smORFs; a machine learning model here such as a neural network is computationally advantageous for learning representations of raw data at this scale across multiple cell unique transcriptomes.

The architecture of the machine learning model may be crafted for adaptability to potential diverse data types that require intermediate representation in the future. In one implementation, the machine learning model is a neural network that includes an initial input layer, covering both DNA and amino acid features in our case, and an output layer with three nodes for classification. See, FIG. 6. Employing the Keras Functional API, the model incorporates crucial components such as batch normalization, activation functions (ReLU), dropout layers, and dense layers. The dataset is divided into training and testing sets with an 80:20 ratio, and hyperparameters are fine-tuned through a randomized search with cross-validation.

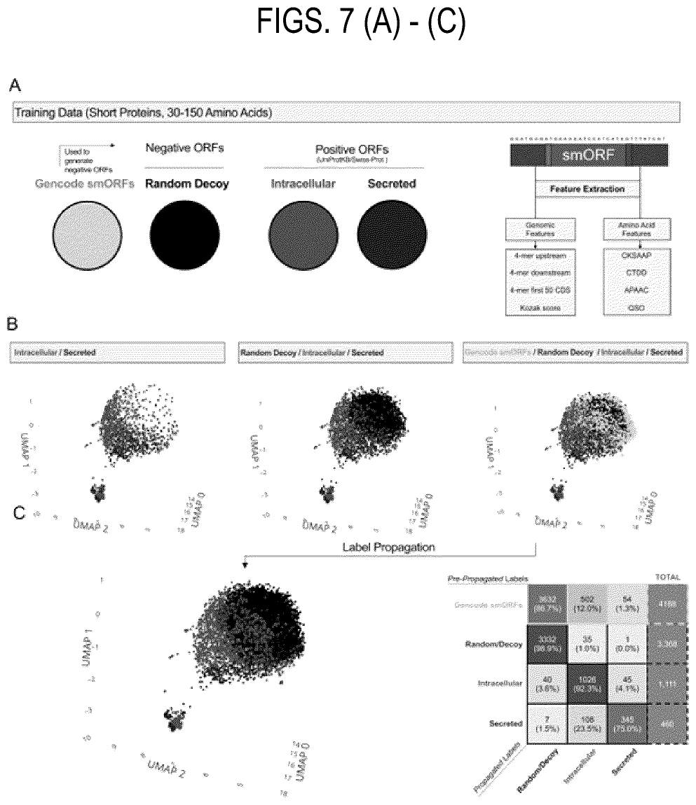

For example, FIG. 7 shows a true positive and negative microprotein set for an exemplary training purpose. In FIG. 7A the true positives were sourced from UniProt with cellular compartments annotated from GO. The positive set was then broken into secreted (blue) and intracellular (red) classes. These short proteins were size selected to include those of 30-150 amino acids in length. The smORFs from GENCODE's Ribo-Seq set were sourced (gold), and then used to generate a random set of proteins, which were treated as true negatives (black). In FIG. 7B, the true positive secreted or intracellular and negative short proteins—as well as the putative smORF GENCODE Ribo-Seq microproteins—were reduced using UMAP following feature reduction. In FIG. 7C, a label spreading algorithm was then deployed, treating the GENCODE Ribo-Seq smORFs as unknowns, and using the known true secreted, intracellular, and negative classes to assign and update labels.

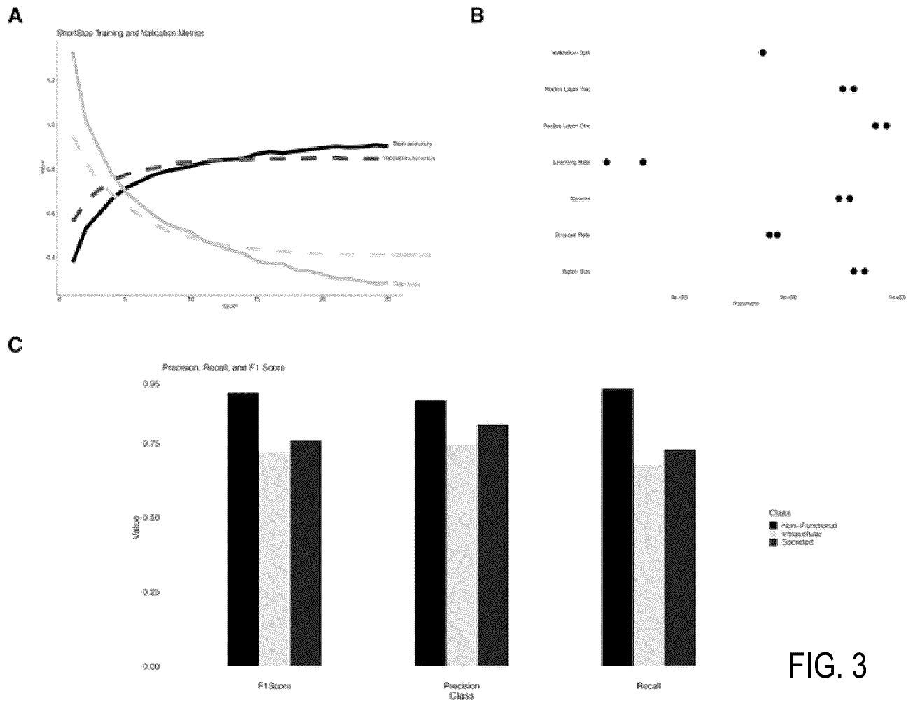

FIG. 3 is a set of graphs showing evaluation metrics for a neural network in accordance with systems and methods described herein. A total of 1,696 features were considered during tuning. In total, following tuning, the model training accuracy was 86.2% with a loss of 0.378 (FIG. 3A-B). During testing, the precision for the microprotein negative class, intracellular class, or secreted class was as follows: 0.89, 0.78, and 0.86. Likewise, the recall was 0.95, 0.68, and 0.68; and the F1 score was 0.92, 0.73, and 0.76 (FIG. 3C).

As an example, the use of a machine learning model trained as described herein can predict negatively annotated microproteins on a deeply sequenced transcriptome derived from HEK293, K562, and HeLa cells. These three cell lines underwent deep RNA-Sequencing as described herein. The advantage of these transcriptomes is that both RNA from both poly A enrichment and RNA depletion was captured, and hundreds of millions of reads were noted for each cell line. As a result, such a machine learning model can predict smORFs not just encoded by high abundant canonical transcripts but also by lower abundant non canonical transcripts. These aligned transcriptome files are fed through a custom in silico, three-frame translation algorithm pipeline to annotate the putative smORFome as described herein. In total, the example machine learning model was used on over one million putative smORFs between 30-150 amino acids from this transcriptome. A primary goal was to identify the quantity and types of smORFs that the machine learning model predicts to be positively annotated (i.e., intracellular or secreted).

The system and method described herein stands out as the first microprotein predictive tool trained on a negative class derived from decoy generation and sub-positive classes of intracellular and secreted proteins. This unique approach makes direct comparisons with previous classification algorithms not feasible, since related algorithms typically focused on annotated transcripts already labeled as “coding” or “noncoding” with no further information about protein localization. In comparison, the question addressed herein is distinctly different: which smORFs look the least like a random decoy counterpart. However, despite the distinctive training methodology, The ability to accurately classify the negative set (F1: 0.95) positions it favorably in comparison to prior algorithms designed for predicting translation initiation (33).

### Application to De Novo Deep-Sequenced Human Transcriptome

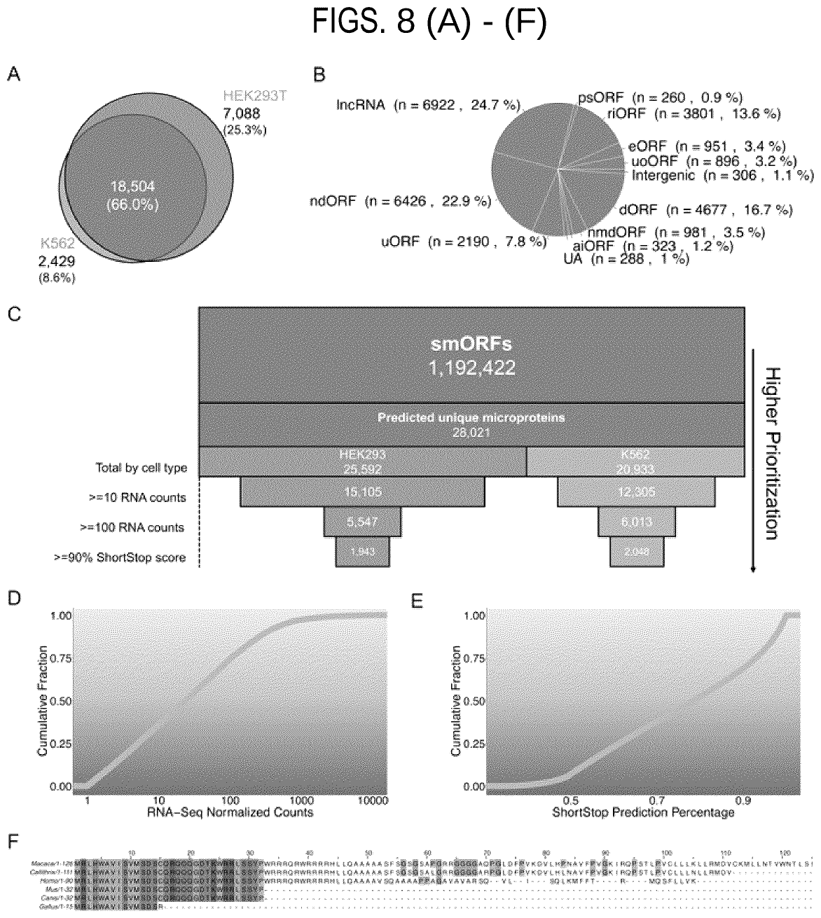

Expanding on the example of FIG. 3 and accompanying text above, FIGS. 8A-8F illustrates in more detail an example of the system and method hereof applied to a deeply sequenced de novo human transcriptome.

FIG. 8(A) is a Venn diagram of the shared number of predicted functionally active microproteins between HEK293T and K562 cells.

FIG. 8 (B) is a Pie graph of the types of smORFs encoding ShortStop predicted functionally active microproteins.

FIG. 8 (C) illustrates the number of predicted functionally active microproteins by cell type and with RNA counts>10, RNA counts>100, and score>90% based on a system and method as described herein (sometimes referred to as “ShortStop”).

FIG. 8 (D) is a graph illustrating the cumulative fraction of predicted microproteins as a function of RNA count, log-scaled. E.g., nearly half of predicted microproteins have RNA counts greater than 100.

FIG. 8 (E) is a graph illustrating the cumulative fraction of predicted microproteins as a function of ShortStop score (prediction percentage for intracellular or secreted positive subclasses).

FIG. 8 (F) Is an example of a predicted microprotein encoded by a smORF on the KATB transcript aligned across multiple species. The number next to the species on the left represents the length of the respective microprotein.

Turning to FIGS. 8 A-F in more detail, one method is particularly valuable for assigning the probability of functional activity to putative smORFs in precious samples. This is relevant when conventional processing methods such as Ribo-Seq or proteogenomics are impractical. As an example a predictive model is applied to a list of putative smORFs extracted from a deeply sequenced transcriptome (GSE125218). This transcriptome, sequenced at a depth exceeding 100 million reads, holds a significant advantage by encompassing both poly-A captured RNA and ribosomal RNA depleted reads across two cell lines: HEK293T, and K562 (11). The extensive sequencing depth enables the comprehensive capture of less abundant transcripts and transcript isoforms, which may potentially encode microproteins.

Out of a vast pool of 1,192,422 putative smORF queries extracted from de novo transcriptome, use of one form of a system and method described herein identified 36,214 unique microproteins. This method was used on a deeply sequenced transcriptome, meaning that many of these transcripts are low in abundance and/or are transcript isoform that do not contain the smORF CDS. Indeed, when counting individual smORF CDS rather than the full transcript, the total count was 28,021. These microproteins exhibited a functional activity probability surpassing 33.4% for either the intracellular or secreted class, constituting 2.4% of the total queries. Furthermore, of the 28, 021 predictions, 23,844 were shared between the HEK293T and K562 cell lines, with 9,380 being unique to HEK293T and 2,812 unique to K562 (FIG. 8A).

The smORF types that were predicted are annotated where defined smORFs are allocated into several categories based on transcript annotation provided by ENSEMBL: retained intron (riORF), non-defined CDS (ndORF), pseudo ORF (psORF), and non-sense mediated decay ORF (nmdORF). In Ribo-Seq, it is common to see nearly three-quarters of total called smORFs annotated upstream in the 5′ UTR of canonical transcripts (3, 11, 14, 27).

The largest category of predicted microproteins was lncRNA-smORFs (24.7%; FIG. 8B). The second largest category was ndORFs (22.9%), which are smORFs encoded by transcripts that lack a canonical CDS; in other words, the main ORF on these transcripts is spliced out. Given that these transcripts are generally lower in abundance, it could explain why some microproteins are also low in abundance or not integrated in the cellular system. It's important to emphasize that, among the total transcripts per protein-coding gene, almost 40% exhibit disrupted main ORF architecture (see Discussion).

While short-read sequencing faces limitations in assigning overlapping reads to distinct transcript isoforms, understanding the number of predicted microproteins encoded by higher-abundance transcripts with countable CDS features. For transcripts with RNA counts exceeding 10 (normalized counts), HEK293T and K562 displayed 15,105 and 12,305 total microprotein predictions, respectively. Raising the count threshold to over 100 resulted in a reduction to 5,547 in HEK293T and 6,013 in K562. And further filtering for microproteins with over 100 RNA counts and over 90% probability, the count was significantly reduced to 1,943 for HEK293T and 2,048 for K562 (FIG. 8C-E). In this scenario, these smORFs might be considered ‘higher priority’ candidates for study due to their abundance and confidence. Overall, these filters effectively reduced the 1,192,422 starting-point query down by 99.8%.

One example of a predicted microprotein appears to have evolved by adding longer c-termini. This microprotein is encoded by a uoORF on the KAT6B transcript. In the species Mus musculus, Canis lupus, and Gallus gallus, this uoORF lacks most of the C-terminus present in Homo sapiens, Rhesus macaque, and Callithrix jacchus. Traditional protein discovery methods emphasizing evolutionary conservation might miss sequences such as this one.

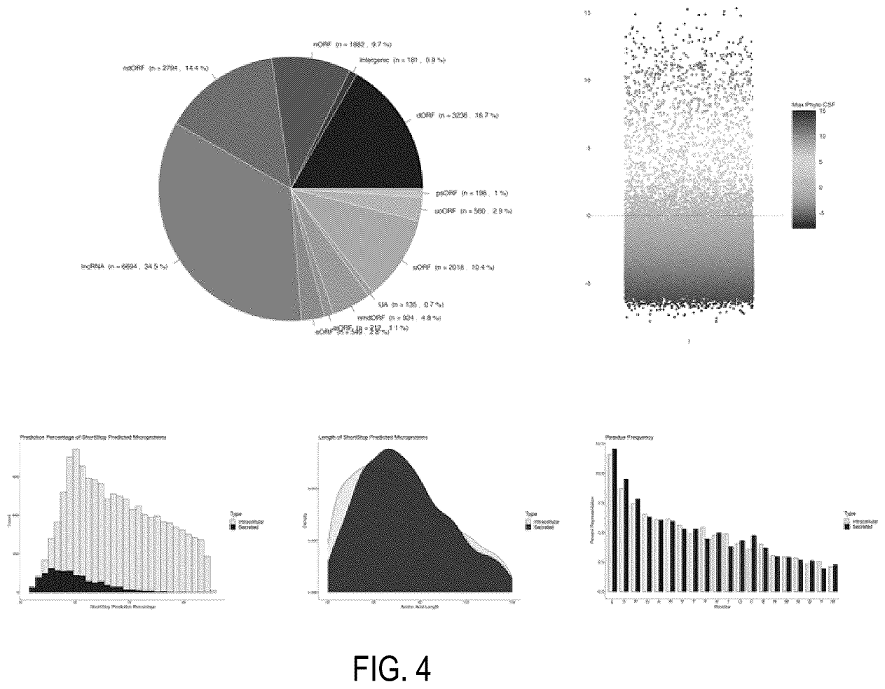

FIG. 4 is another example of using a machine learning model trained as described herein to predict annotated microproteins on a deeply sequenced transcriptome derived from HEK293, K562 and HeLa cells. From a total of 1,007,499 ORF queries derived from the HEK293/HeLa/K562 transcriptome, a trained neural network predicted 19,408 unique smORFs to encode positively annotated microproteins with a probability percentage over 33.4% (1.93%). Many of these unique smORFs encode have isoforms—in total, when considering isoforms, The trained neural network predicted 46,064 smORFs with a probability percentage over 33.4% (4.57%). For the 19,408 unique smORFs (taking just one of its isoforms from the 46,064 set), the majority are encoded by transcripts annotated as ‘long non-coding RNA’ (6,694; 34.5%; FIG. 4A). Depending on how smORFs are annotated, the next largest class of smORFs reside in the 3′ downstream untranslated region (dORF) of known protein-coding genes (3,326; 16.7%). The next classes of smORFs reside in transcripts with non-defined protein coding regions (2,794; 14.4%) or 5′ upstream untranslated regions uORFs (2018, 10.4%). The rest of predicted microproteins from smORFs (<10%) reside in transcripts with retained introns, overlapping exonic regions of protein-coding genes, non-sense mediated decay transcripts, alternative initiation within the first exon of a protein-coding gene, upstream overlapping regions of the first CDS of a protein-coding gene, or novel transcripts/low abundant transcripts in intergenic regions. Most of these smORFs are not well-conserved and fall below a maximum PhyloCSF score below 0 (FIG. 4B).

By applying a stricter cutoff of greater than or equal to 50% or 70%, respectively, 14,785 (1.47%) and 6,839 (0.69%) smORFs were predicted to encode a microprotein that falls into the intracellular or secreted class. Most predicted microproteins fell within the intracellular class (FIG. 4C); of the 6,839 predicted microproteins over 70%, just 69 were predicted into the secreted class, whereas 6,770 were predicted into the intracellular class (FIG. 4C). Both the intracellular and secreted class of predicted microproteins have the same amino acid length distribution, peaking at approximately 75 amino acids in length (FIG. 4D), as well as similar amino acid frequency (FIG. 4E). As has been previously noted for microproteins, leucine (12%), serine (9%), proline (8%), and glycine (6%) are particularly abundant in microproteins predicted by the trained machine learning model hereof Nevertheless, there was a slight increase in frequency for leucine, serine, and proline by about ˜1% for sequences predicted in the secreted class, with cysteine frequency being the starkest between the classes and particularly higher in the secreted class (˜1.5%).

The systems and methods described herein have a number of advantages. First, Ribo-Seq with deep transcriptome sequencing costs $15,000-$20,000 and at minimum 2 months to complete with highly experienced bench scientists and bioinformaticians. Second, a CRISPR screen against 8,000 smORFs also $15,000 and takes at minimum 6 months to complete. Only cell survival and 1-2 additional phenotypes can be tested at the same time. Therefore, deep transcriptome assembly+Ribo-Seq+CRISPR screens costs $30,000-35,000 and, with everything running smoothly, can be completed within one year. The systems and methods described herein have little cost, can be run on a personal laptop, and takes minutes, not months.

The systems and methods described herein are additionally advantageous in that unlike Ribo-Seq, systems and methods described herein do not care about repetitive regions. Unlike gene prediction algorithms, the systems and methods described herein do not care about evolutionary conservation. Unlike other short protein prediction algorithms, the systems and methods described considers a genuine “negative” label class. The systems and methods described herein can be applied to previous samples without the need for more sample processing (e.g., cancer tumors, etc.) and is not restricted by high volume of samples.

Further analysis can be performed using various cell types such as HEK293 cells, K562 cells, and cells used in immunological analyses such as cancer types and immune compatibility (e.g., Cancer HLA cells, optionally used to generate a Venn diagram, or healthy/diseases cells having various expression patterns of human leukocyte antigen (HLA)).

### Peptide-Level Evidence of Predicted Microproteins

The role of alternative translation of smORFs in cancer has garnered recent interest. Several smORFs found within annotated lncRNAs or upstream regions of canonical genes have demonstrated significance in cancer proliferation. And deep proteomic sequencing has unveiled tumor-specific immunopeptidomes containing non-canonical peptides. In cancer research, a major challenge lies in profiling the genomic diversity driven by mutation burdens.

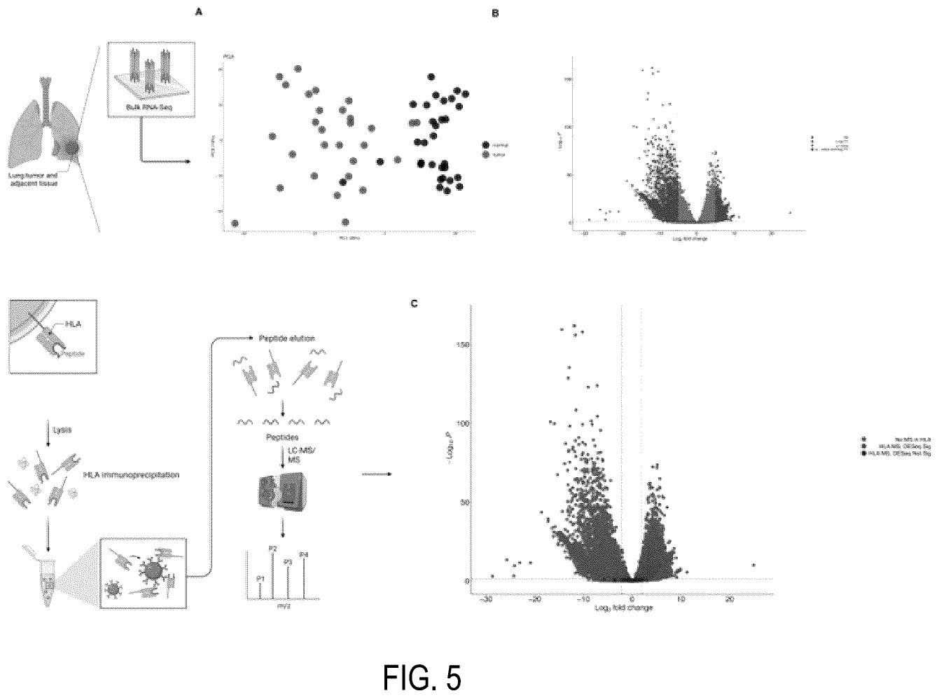

A trained machine learning model in accordance with the described embodiment can potentially identify positively annotated microprotein smORFs displaying differential expression in cancer. To test this, publicly available bulk RNA-Seq data from a cohort of lung cancer patients who had never smoked was analyzed. Specifically, the raw fastq files from 31 normal and 29 adjacent tissue samples were realigned, and then differential expression analysis conducted on counts of smORFs predicted as positively annotated by the trained machine learning network (with a prediction probability>33.3%). After dimension reduction through principal component analysis (PCA), two distinct clusters are present, clearly separating tumor and adjacent matched tissue samples (see FIG. 5A). This finding suggests that the transcripts on which the trained machine learning model predicts positively annotated microproteins exhibit significant differences in tumors, thus supporting a hypothesis that the trained machine learning model can identify positively annotated microprotein smORFs displaying differentiated expression in cancer. Intriguingly, the majority of predicted positively annotated microproteins that exhibited differences in tumors were down-regulated, with many showing substantial log fold changes below −5.0.

A critical consideration in these differential expression analyses is that many of these transcripts may have functions beyond potentially encoding positively annotated microproteins (such as uORFs, dORFs, lncRNAs, etc.). See FIG. 5. To address this concern, proteomics analyses of HLA complexes isolated from lung cancer samples can be conducted. In theory this leads to a significant number of peptide spectrum matches (PSMs) enriched in tumors. Indeed, analysis of the raw proteomics data identifies a total of 434 microproteins with at least one confidently matched peptide (FDR<0.01). Among these 434 microproteins, 346 display differential expression in cancer at the RNA level (FDR 0.01). Notably, an enrichment of detectable microproteins in HLA complexes that were upregulated at the RNA level (p<1e-5) is observed. This suggests the possibility that certain microproteins play a bioactive, prominent role in cancer biology and could potentially serve as novel targets for cancer therapy due to their presentation from HLA complexes.

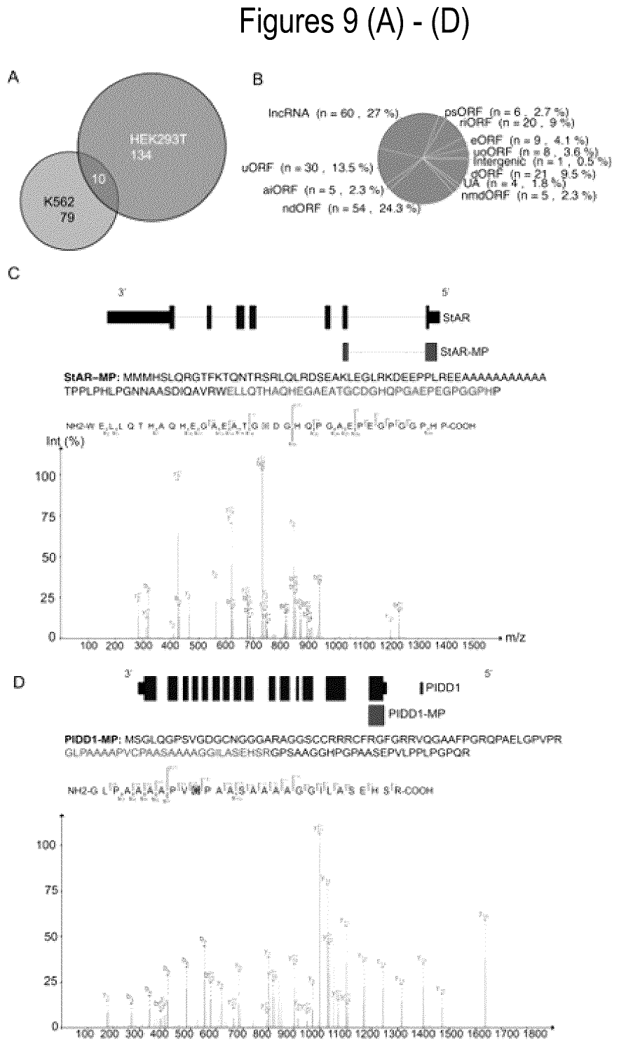

FIG. 9 broadly illustrates peptide-level detection of predicted microproteins in HEK293T and K562 cell lysates where:

FIG. 9(A) Venn Diagram of the predicted active microproteins in MS experiments of low molecular weight enriched HEK293T and K562 cell lysates;

FIG. 9 (B) Pie graph of the types of smORFs with peptide-level detection in HEK293T and K562 cells lysates.

FIG. 9 (C-D) are graphs showing detection of unique tryptic peptides (MS/MS spectrum shown) for two predicted microproteins. From top to bottom is StAR-MP (uoORF) and PIDD1-MP (uoORF).

Previous studies demonstrate the effectiveness of a proteogenomics approach to discover intracellular microproteins, notably CYREN, NoBody, and PIGBOS (9, 10, 45). However, proteogenomics is challenged by a vast search space of smORFs containing upwards to 10-15 million sequences (46). To address this challenge, a proteogenomics hybrid approach is employed to identify microproteins in K562 cells and HEK293T cells. Instead of including the complete smORF list in the search space during mass spectrometry analysis, the predicted microproteins are incorporated along with reference human proteome and common peptide contaminants. Additionally, the cell lysates are prepared of K562 and HEK293T cells by implementing a low molecular weight enrichment strategies. The sample is prepared to optimally detect lower abundant, smaller proteins.

Following sample preparation and mass spectrometry, two significant cutoffs are applied when analyzing the mass spectra: an FDR of 0.05 and FDR of 0.2 at the peptide level. The reasoning for the two FDR cutoffs is that the larger search space, as a result, might lead to conservative calling. Thus, the data are analyzed using a rather relaxed method (FDR of 0.2) and more conservative methods (FDR of 0.05). Analyzing the data using an FDR of 0.05 identifies 89 unique peptide fragments that mapped to a unique microproteins in K562 cells and 144 microproteins in HEK293T cells, with 10 microproteins shared between both cell types (FIG. 9A). Among these 223 microproteins, 60 are encoded by smORFs within lncRNAs, representing the predominant smORF type. The second most common smORF type was ndORF (54), followed by uORFs (30) and dORFs (21) (FIG. 9B). Notably, there were no differences in the tryptic sequences of microproteins called with an FDR of 0.05 versus FDR of 0.2, other than total number of PSMs.

One goal is to assess the probability percentage associated with MS detection. To address this, MS is modeled as a binary variable and the prediction percentage is used as an independent variable in a logistic regression model. Indeed, there was a very modest and minimally significant effect of prediction percentage on MS detection (log-odds: 1.69, p<0.05; FIG. 9).

Focusing on upstream overlapping ORFs (uoORFs), mass spectrometry evidence is illustrated for a uoORF on the Steroidogenic Acute Regulatory (StAR) protein transcript and a separate uoORF on the PIDD1 transcript (FIG. 9C-D) with visually confirmed quality spectra. Microproteins encoded by uoORFs are particularly intriguing. These uoORFs can be challenging to detect in Ribo-Seq because they overlap a canonical ORF. They also must undergo translation that prioritizes the upstream start codon over the canonical CDS start codon. The mechanism governing this mode of translation remains unresolved, suggesting either a novel form of translation or the presence of post-transcriptional regulation yielding unique transcript isoforms. While the detection of a microprotein using mass spectrometry does not directly indicate activity, these experiments suggest that the detected microproteins have a high probability of integration into the cellular system, given both their detection and prediction assignment by an exemplary system and method hereof.

### Feature Characteristics of Predicted Microproteins

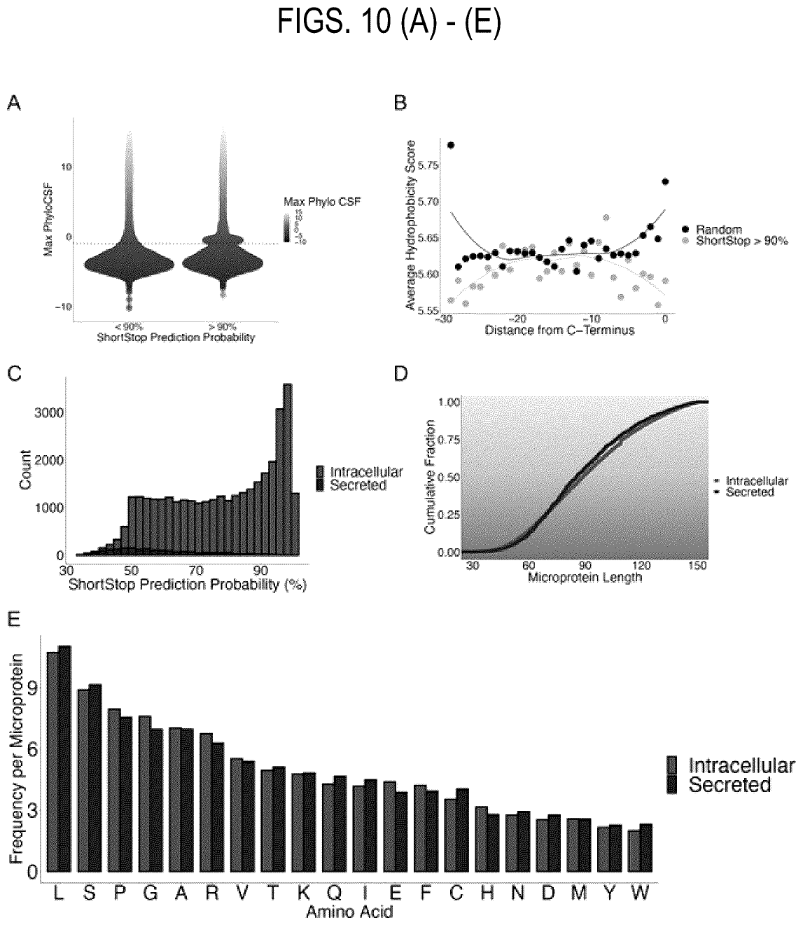

FIGS. 10(A)-(E) illustrates the feature characteristics of higher confident predictions and subclasses predictions.

FIG. 10(A) is a violin plot representing the distribution of microproteins according to each max PhyloCSF score (y axis), which is also color-coded by max PhyloCSF. The two violins represent microproteins with a predicted positive functional probability score less than 90% (left) and greater than 90%.

FIG. 10(B) shows C-terminal hydrophobicity of predicted microproteins: the X axis represents the amino acid distance from the C-terminus. Each point represents the average hydrophobicity of the predicted microprotein class at the specific residue distance from the C-terminus. Black points and line represent the random/decoy class, whereas gold points and line represents the functional class with a greater than 90% probability functional score.

FIG. 10(C) Prediction percentage histogram of predicted active microproteins: the X axis represents the total count, and the Y axis represents the probability assignment by the present method, separated by intracellular (red) and secreted (blue) classes.

FIG. 10(D) Length of predicted microproteins: the Y axis represents the cumulative fraction of all microproteins predicted in the intracellular (red) or secreted (blue) class, and the X axis represents the length of the sequence.

FIG. 10(E) Residue frequency per microprotein for the intracellular and secreted predicted classes.

The correlation between higher-confidence predicted microproteins (i.e., smORFs with a >=90% score) and their level of evolutionary conservation is worth considering. Although the difference seemed subtle, microproteins with a confidence level exceeding 90% exhibited an average maximum PhyloCSF score of −2.25, in contrast to −2.70 for those with less than 90% probability (p<2e-16) (FIG. 5A). To further understand predictions, the impact of hydrophobic C-termini selection in proteins across evolutionary ages (19) is considered. Analyzing the 90% microprotein set, lower hydrophobicity per residue is observed compared to the random/decoy class (FIG. 10B). These collective findings suggest that the present method assigns higher confidence to predictions that show slightly higher conservation or possess biochemical features akin to proteins with the potential to emerge.

When classifying intracellular versus secreted subclasses of short proteins, 94.8% of all predictions were intracellular, and this percentage further rose to 99.3% when considering microproteins with a probability exceeding 90% (FIG. 10C). Additionally, the amino acid composition and length of these intracellular microproteins did not significantly differ from those predicted to be secreted (FIG. 10D-E). Leucine emerged as the most frequently used amino acid in both classes, aligning with previous reports on actively translated microproteins via Ribo-Seq (11). Notably, the length distribution of predicted microproteins revealed that approximately 75% were under 100 amino acids, with 25% being under 75 amino acids, as illustrated in FIG. 10D.

### Predicted Microproteins in Cancer and as Immunopeptides

Building on the analysis and discussion of FIG. 5, one form of the system and method hereof was applied to lung cancer, specifically the Sherlock-Lung study.

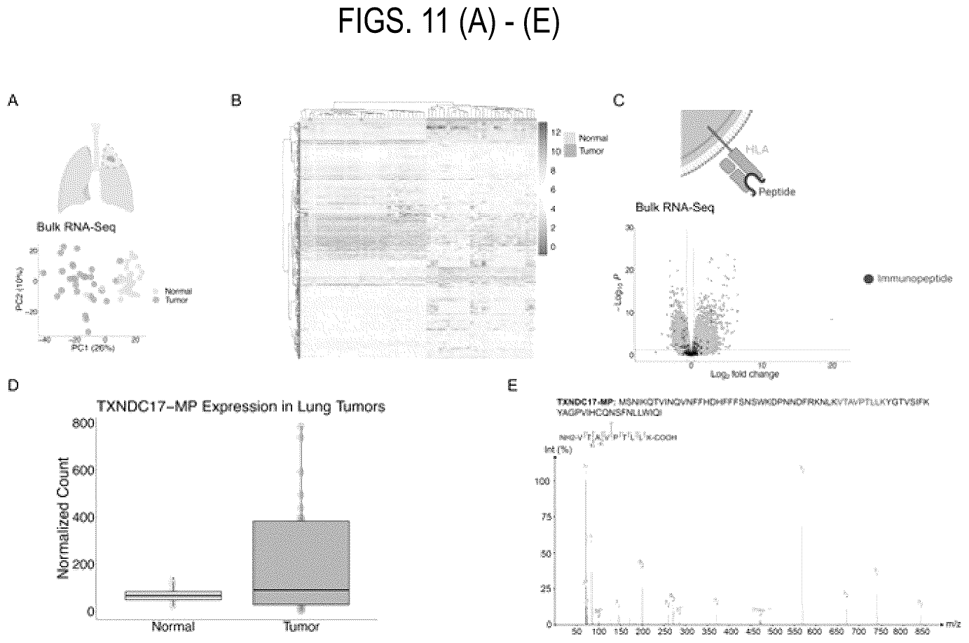

FIGS. 11 (A)-(E) relate to the RNA expression of predicted microproteins and immunopeptidomic detection in lung cancer.

FIG. 11(A) Bulk RNA-Seq re-analysis of lung cancer in never smokers (LCINS) from the Sherlock-Lung study. The scatter plot represents a PCA (x axis is PC1, and y axis is PC2) of the count data; tan points represent normal lung tissue and pink points represent lung tumor samples.

FIG. 11 (B) Hierarchical clustering plot of the first 500 most significantly differentially expressed microprotein smORFs between tumors and normal tissue samples.

FIG. 11 (C) Immunopeptidomics re-analysis of two pairs of lung cancer samples with matched normal tissues of HLA class-1 bound peptides. A volcano plot from the RNA-Seq analysis in A-B is used here with each point/microprotein color coded by its detection via immunopeptidomics (red).

FIG. 11 (D) Box plot representation of TXNDC17-MP in normal vs. tumor samples

FIG. 11 (E) MS2 of TXNDC17-MP detected as tumor HLA1 immunopeptides, with the detected fragment color-coded in red.

It has been reported that non-canonical proteins are enriched in the MHC-1 immunopeptidome and could serve as potential cancer therapeutic targets. For instance, Cuevas et al. portrayed that a substantial portion of the proteins detected in MHC-1 complexes were encoded by smORFs and non-canonical ORFs (44). However, whether these non-canonical proteins are functional remains unclear. While these non-canonical peptides are hypothesized to serve as immunosurveillance factors, it is also plausible that some may be integrated into the cellular system, carrying implications for cancer survival distinct from their role as immunosurveillance factors.

The system and method hereof can identify differentially expressed microproteins in lung tumors. To illustrate, publicly available bulk RNA-Seq data from the Sherlock-Lung study is analysed, involving 29 non-smoking lung cancer patients. This data contains RNA reads from lung tumors and matched normal lung tissue. A differential expression analysis on counts of smORF exons predicted from the microprotein set revealed two distinct clusters through principal component analysis (PCA), effectively separating tumor and adjacent matched normal tissue samples (FIG. 11A). This clustering indicated significant transcript differences in tumors at both upregulation and downregulation levels (FIG. 11B). In total, transcripts with 12,273 predicted smORFs were differentially expressed between tumors and normal tissue at an FDR of 0.2. Notably, the largest fold changes tended to be in upregulation in tumors. Among 819 smORFs with a log 2 fold change under two or over two, 649 displayed a log 2 fold change over two (representing 79.2%). This underscores the cryptic nature of microproteins in tumors that, for extreme changes, appeared mostly upregulated.

Another possibility is that many of these differentially expressed smORFs in cancers could encode microproteins that are loaded onto MHC1 complexes. Upon re-analyzing mass spectra data of HLA complexes isolated from two pairs of lung cancer samples with matched normal tissues, a total of 226 microproteins were counted that were detectable as immunopeptides. Notably, of these 226 microproteins, differentially expressed at the RNA level between tumors and normal tissue from the Sherlock-Lung study (FIG. 11C). Among these, one noteworthy microprotein encoded by a ndORF within an TXNDC17 transcript isoform ENST00000574429.1. This demonstrates that this microprotein is substantially upregulated in tumors from the Sherlock-Lung study (FIG. 11D), and its microprotein is a detected byproduct via MS (PXD013649) (FIG. 11E).

### Endogenous Detection of Predicted Microprotein Encoded by the StAR Transcript (StAR-MP)

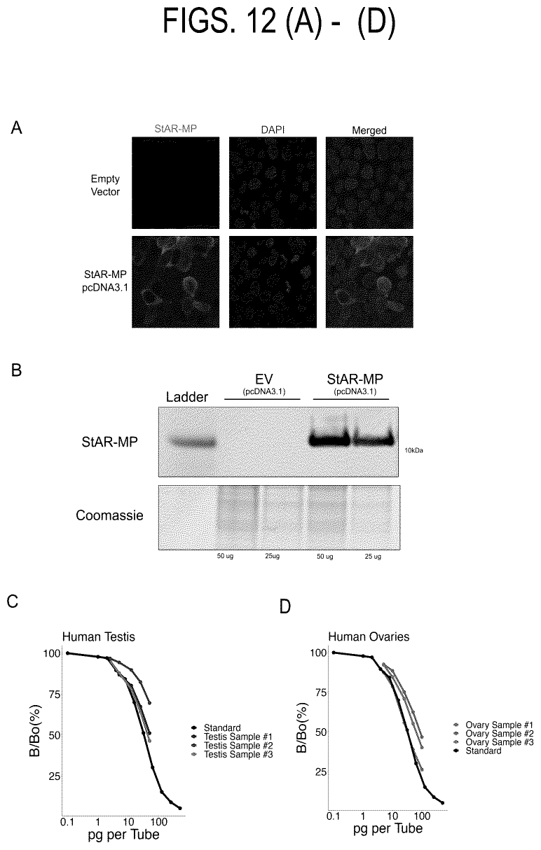

FIG. 12(A)-(D). Highly specific antiserum raised in rabbit using a synthetic peptide fragment of StAR-MP

FIG. 12(A) Immunofluorescence staining of HEK293T cells transiently transfected with a StAR-MP construct, visualized with the anti-StAR-MP serum followed by a goat anti-rabbit IgG highly cross-absorbed secondary antibody Alexa488. Cells were counterstained with DAPI to visualize transfected and control transfected cells.

FIG. 12 (B) Western immunoblot of StAR-MP from HEK293T cell lysates transiently transfected with control construct (left two lanes; 50 ug, 25 ug) or with anti-StAR-MP construct (far right two lanes; 50 ug, 25 ug). Bottom blot indicates total protein transferred as evident by Coomassie staining.

FIG. 12 (C-D) Displacement of [125I-Tyr38]-StAR-MP(4-38) peptide binding to rabbit antiserum against StAR-MP by synthetic StAR-MP(4-38) peptide and, from left to right, human testis extracts and human ovary extracts. The Y axis is the B/Bo (%), and the X axis is the total amount of protein in pg.

While the present system and method successfully detected predicted microproteins in cell lysates another objective was to examine the stability and endogenous abundance in relevant tissues. Focusing on StAR-MP due to its high-confidence spectra in the proteomics of K562 cell lysate fractions and its downstream role in steroidogenesis through its CDS (StAR) (47). To investigate the endogenous levels of StAR-MP, a specific antiserum against StAR-MP is generated and employed to study StAR-MP expression in cells and tissues.

First, overexpressing a StAR cDNA construct in HEK293T cells. Unlike many microprotein studies involving fusion tags, not tagging StAR-MP preserves its native conformation and stability. Fusion tags could lead to mislocalization and affect stability; for instance, commonly used C-terminal FLAG tags may cause mislocalization. Therefore, overexpressing untagged StAR-MP and analyzing its stability with custom antiserum best reflects its potential bioactivity. After 24 hours of overexpression in HEK293T cells, StAR-MP was consistently detected in whole cells by immunofluorescence and in cell lysates by Western blot in a dose-dependent manner (FIGS. 12A-B). Notably, HEK293T cells lack natural expression of the StAR transcript, making them a suitable negative control. StAR-MP was not detected in cells transfected with an empty vector, confirming its presence only with overexpression.

Second, analyzing endogenous levels of StAR-MP in tissues expressing its transcript. Studying human testis and ovary extracts from six donors, chosen for their high StAR expression and relevance to steroidogenesis. Using a sensitive StAR-MP radioimmunoassay (RIA) StAR-MP in six donor samples of normal human testis and ovaries is measured, indicating its endogenous expression (FIG. 12C). Note the expression of StAR-MP using this RIA in cell lines, noting its absence in HEK293T cells that don't express its transcript compared to steroidogeneic-producing cells and cancer cells that do express its transcript (Supplemental FIG. 6). These findings suggest that StAR-MP is a stable microprotein expressed in steroid producing tissues. Its precise biological function and regulation of StAR protein itself warrant further study.

FIG. 13 is a flow diagram of one embodiment of a method for training a machine learning model to classify unknown amino acid sequences. Method 1300 may be performed by any suitable computer system, such as that described below with respect to FIG. 15. Method 1300 has many variations, including those described below.

Method 1300 includes, at 1310, accessing a set of data describing expressed amino acid sequences. As used herein, “expressed” amino acid sequences includes those amino acid sequences known to be expressed by an organism, as well as those amino acid sequences that can be predicted to expressed by an organism. In one embodiment, the set of data includes information describing unlabeled amino acid sequences having unknown classifications. This set of data may, for example, include Ribo-Seq derived ORFs, including GENCODE small open reading frames (smORFs) having unknown classifications. In other embodiments, the set of data may include amino acid sequences having known classifications.

Method 1300 continues in 1320, with generating decoy training data that includes randomized amino acid sequences that are matched to properties of the amino acid sequences included in the set of data. In some embodiments, the randomized amino acid sequences included in the decoy training data are matched to lengths and codon probabilities of the expressed amino acid sequences included in the set of data. The decoy training data includes amino acid sequences that are not expected to fall into one of a set of classifications, and thus is intended to constitute negative training examples.

Method 1300 concludes in 1330, with training a machine learning model using labeled training data and the decoy training data, the labeled training data including amino acid sequences within a set of classifications and the decoy training data constituting examples of amino acid sequences that are not expected to be within the set of classifications. One example of labeled training data is a list of proteins from Swiss-Prot. The trained machine learning model produced by method 1300 is usable to receive an input that describes a structure of a particular amino acid sequence, and perform a classification of the particular amino acid sequence relative to the set of classifications. In some embodiments, performing the classification of the particular amino acid sequence relative to the set of classifications includes generating probabilities that the particular amino acid sequence is in one or more of the set of classifications (e.g., where there are four possible classifications, up to four percentages or probabilities might be generated). In other embodiments, performing the classification of the particular amino acid sequence relative to the set of classifications may simply include selecting one classification within the set of classifications.

The set of classifications may include any suitable classifications. For example, one possible set of classifications includes intracellular, secreted, and negative. In some embodiments, the set of classifications may be user-specified and configurable, so as to meet a particular user's needs.

In some embodiments, method 1300 may include label spreading, with the method further comprising performing feature extraction on amino acid sequences described in the labeled training data and the decoy training data, and labeling, based on information derived from the feature extraction, the unlabeled amino acid sequences in the set of data to create additional labeled training data. The machine learning model is also trained using the additional labeled training data. The extracted features resulting from the feature extraction may include nucleotide features and amino acid features in some embodiments. The nucleotide features may include one or more of 4-mers of the 5′ UTR, 3′ UTR, first 50 CDS. The amino acid features, on the other hand, may include one or more of CTDD, CTD, APAAC, QSO.

Method 1300 may further comprise use of the model. Thus, method 1300 may include receiving, by the trained machine learning model, a description of the particular amino acid sequence, and then the trained machine learning model performing a classification of the particular amino acid sequence relative to the set of classifications. In some scenarios, a model may be trained on one computer system and used on another computer system.

Method 1300 may be of particular use in classifying short amino acid sequences that have historically overlooked as having significance, including those of 300 amino acids or less, 150 amino acids or less, 100 amino acids or less, etc. Method 1300 is thus of particular use with respect to microproteins.

Method 1300 has a number of possible variations. One such variation includes accessing labeled training data that describes amino acid sequences with known classifications, and generating decoy training data that includes randomized amino acid sequences with properties that are matched to properties of actual amino acid sequences, the decoy training data constituting negative training examples. The variation concludes with training a machine learning model using the labeled training data, and the decoy training data, the trained machine learning model being usable to classify unknown amino acid sequences into one of a set of classifications.

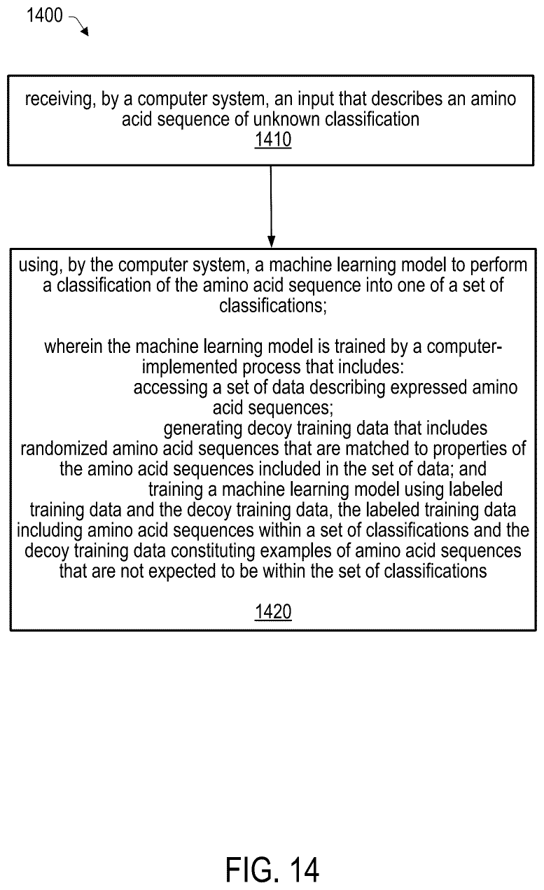

FIG. 14 is a flow diagram of one embodiment of using a trained machine learning model to classify unknown amino acid sequences. Method 1400 may be performed by any suitable computer system, such as that described below with respect to FIG. 15. Method 1400 has numerous variations.

Method 1400 includes, in 1410, receiving an input that describes an amino acid sequence of unknown classification, and in 1420, using a machine learning model to perform a classification of the protein into one of a set of classifications. The machine learning model is trained by a computer-implemented process that includes:
accessing a set of data describing expressed amino acid sequences;
generating decoy training data that includes randomized amino acid sequences that are matched to properties of the amino acid sequences included in the set of data; and
training a machine learning model using labeled training data and the decoy training data, the labeled training data including amino acid sequences within a set of classifications and the decoy training data constituting examples of amino acid sequences that are not expected to be within the set of classifications. The randomized amino acid sequences included in the decoy training data may be matched to lengths and codon probabilities of the actual amino acid sequences.

Various techniques described herein, may be performed by one or more computer programs. The term “program” is to be construed broadly to cover a sequence of instructions in a programming language that a computing device can execute or interpret. These programs may be written in any suitable computer language, including lower-level languages such as assembly and higher-level languages such as Python.

Program instructions may be stored on a “non-transitory, computer-readable storage medium” or a “non-transitory, computer-readable medium.” The storage of program instructions on such media permits execution of the program instructions by a computer system. These are broad terms intended to cover any type of computer memory or storage device that is capable of storing program instructions. The term “non-transitory,” as is understood, refers to a tangible medium. Note that the program instructions may be stored on the medium in various formats (source code, compiled code, etc.).

The phrases “computer-readable storage medium” and “computer-readable medium” are intended to refer to both a storage medium within a computer system as well as a removable medium such as a CD-ROM, memory stick, or portable hard drive. The phrases cover any type of volatile memory within a computer system including DRAM, DDR RAM, SRAM, EDO RAM, Rambus RAM, etc., as well as non-volatile memory such as magnetic media, e.g., a hard drive, or optical storage. The phrases are explicitly intended to cover the memory of a server that facilitates downloading of program instructions, the memories within any intermediate computer system involved in the download, as well as the memories of all destination computing devices. Still further, the phrases are intended to cover combinations of different types of memories.

In addition, a computer-readable medium or storage medium may be located in a first set of one or more computer systems in which the programs are executed, as well as in a second set of one or more computer systems which connect to the first set over a network. In the latter instance, the second set of computer systems may provide program instructions to the first set of computer systems for execution. In short, the phrases “computer-readable storage medium” and “computer-readable medium” may include two or more media that may reside in different locations, e.g., in different computers that are connected over a network.

Note that in some cases, program instructions may be stored on a storage medium but not enabled to execute in a particular computing environment. For example, a particular computing environment (e.g., a first computer system) may have a parameter set that disables program instructions that are nonetheless resident on a storage medium of the first computer system. The recitation that these stored program instructions are “capable” of being executed is intended to account for and cover this possibility. Stated another way, program instructions stored on a computer-readable medium can be said to “executable” to perform certain functionality, whether or not current software configuration parameters permit such execution. Executability means that when and if the instructions are executed, they perform the functionality in question.

Similarly, systems that implement the methods described with respect to any of the disclosed techniques are also contemplated. One such system is described with reference to FIG. 15. FIG. 15 is a block diagram of another embodiment of such a computer system. Computer system 1500 includes a processor subsystem 1580 that is coupled to a system memory 1520 and I/O interfaces(s) 1540 via an interconnect 1560 (e.g., a system bus). I/O interface(s) 1540 is coupled to one or more I/O devices 1550. Computer system 1500 may be any of various types of devices, including, but not limited to, a server system, personal computer system, desktop computer, laptop or notebook computer, mainframe computer system, tablet computer, handheld computer, workstation, network computer, a consumer device such as a mobile phone, music player, or personal data assistant (PDA). Although a single computer system 1500 is shown in FIG. 15 for convenience, system 1500 may also be implemented as two or more computer systems operating together.

Processor subsystem 1580 may include one or more processors or processing units. In various embodiments of computer system 1500, multiple instances of processor subsystem 1580 may be coupled to interconnect 1560. In various embodiments, processor subsystem 1580 (or each processor unit within 1580) may contain a cache or other form of on-board memory.

System memory 1520 is usable to store program instructions executable by processor subsystem 1580 to cause system 1500 perform various operations described herein. System memory 1520 may be implemented using different physical memory media, such as hard disk storage, floppy disk storage, removable disk storage, flash memory, random access memory (RAM-SRAM, EDO RAM, SDRAM, DDR SDRAM, RAMBUS RAM, etc.), read only memory (PROM, EEPROM, etc.), and so on. Memory in computer system 1500 is not limited to primary storage such as memory 1520. Rather, computer system 1500 may also include other forms of storage such as cache memory in processor subsystem 1580 and secondary storage on I/O Devices 1550 (e.g., a hard drive, storage array, etc.). In some embodiments, these other forms of storage may also store program instructions executable by processor subsystem 1580.

I/O interfaces 1540 may be any of various types of interfaces configured to couple to and communicate with other devices, according to various embodiments. In one embodiment, I/O interface 1540 is a bridge chip (e.g., Southbridge) from a front-side to one or more back-side buses. I/O interfaces 1540 may be coupled to one or more I/O devices 1550 via one or more corresponding buses or other interfaces. Examples of I/O devices 1550 include storage devices (hard drive, optical drive, removable flash drive, storage array, SAN, or their associated controller), network interface devices (e.g., to a local or wide-area network), or other devices (e.g., graphics, user interface devices, etc.). In one embodiment, computer system 1500 is coupled to a network via a network interface device 1550 (e.g., configured to communicate over WiFi, Bluetooth, Ethernet, etc.).

Memory 1520 may include a non-transitory computer-readable storage medium storing program instructions 1522 in various embodiments. Program instructions 1522 may include instructions that are executable to perform methods 1300-1400, for example.

### General Discussion

A primary goal of the system and method described herein is to establish a new tool to complement existing microprotein discovery approaches. The system and method described herein is designed to differentiate putative microproteins from random generated counterparts. The system and method hereof is a computational experimental platform that takes putative sets of microproteins and then generates an equally matched set of random/decoy ORFs based on amino acid probability, length distribution, and nucleotide frequency. Then, a positive set of experimentally validated proteins is input during training. This way, the system and method hereof generates a predictive model that classifies putative microproteins as “random/decoy-like” or “experimentally characterized-like” proteins. Users have flexibility over what positive labels are ultimately used.

Many challenges are posed by Ribo-Seq and proteogenomics. For example, Ribo-Seq may overlook smORFs containing repetitive sequences, as the reads being approximately 30 nucleotides long struggle with proper alignment to a reference genome (48). Even for smORFs that align accurately, it remains uncertain whether they encode functionally active microproteins. In many cases, the active translation of a smORF might serve as a cis-regulator for a downstream ORF (e.g., uORFs such as AT4) (49). In relation to proteogenomics, a significant limitation is the ionization potential for short proteins. Because they have fewer enzymatic sites (e.g., trypsin) compared to larger molecular weight proteins, microproteins are inherently less likely to be identified by traditional MS workflows. Even if microproteins possess enzymatic sites, they might not ionize effectively or may lack sufficient abundance for detection. These biochemical challenges are further complicated by a large protein identification search space during analysis, sometimes consisting of millions of sequences in the search space. This expanded search space can lead to higher false negatives due to misalignment of spectra, necessitating statistical corrections for millions of entries and demanding enormous computational resources (46).

The system and method hereof stands out as the first microprotein prediction tool to implement this decoy strategy. The system and method described herein generates a “standard predictive model.” Ribo-Seq data is used from the GENCODE smORF Phase 1 catalog (12). Since these smORFs were highly scrutinized for their translational activity, a matched random set of smORFs is generated in terms of their length distribution and amino acid probability, borrowing from past experimental designs to study de novo emergence of proteins (30). With the establishment of a true negative list, short proteins were sourced (30-150 amino acids) from UniProt/Swiss-Prot with cellular compartment annotations, designating them as “true positives.” As a result, the system and method hereof utilizes a set of true negatives and positives to train models.

To reduce the dimensionality of feature space, UMAP is employed. The primary goal of UMAP is visual exploration to identify patterns among microproteins initially labeled as “random/decoy, GENCODE smORFs, intracellular, or secreted.” Distinctive visual patterns emerged during analysis. Notably, the random/decoy microprotein set formed a cluster that nearly overlapped with the GENCODE smORF set. This suggests that the majority of microproteins encoded by active-translating smORFs share features of randomly generated microproteins. Additionally, short proteins annotated as intracellular or secreted clustered distinctly, suggesting that not only do feature reductions in this reduced space portray differences between random/decoys but also between intracellular and secreted classes. Interestingly, hundreds of GENCODE smORFs, while mostly clustering with the random/decoy class, deviated from the random/decoy focal point and appear to cluster with the positively treated intracellular and secreted proteins.

Based on UMAP visualization, a label propagation step is used to maximize the information provided by GENCODE smORFs. To do this, GENCODE smORFs are treated as unknown (i.e., neither being negative nor positive), thereby letting the known labels of random/decoy, intracellular, and secreted “spread” akin to viral infection in this 3D UMAP space. Following label propagation, ˜85% of the GENCODE smORFs were re-labeled as “random/decoy.” UMAP does not guarantee that distances between points in reduced space accurately represent distances in the original data. Users should exercise caution when inferring distances in reduced space. Nevertheless, 98.9% of the original random/decoy labels retained their initial designation following label propagation, preserving essentially all the original labels of microproteins for downstream training in higher-dimensional space. A user might simply bypass the label propagation step after generating random/decoy microproteins. Preferably the downstream model is trained on these propagated labels to maximize the information gained from GENCODE smORFs.

In one embodiment, with a negative and positive set of microproteins, a basic neural net is trained using the 1,631 features to classify random/decoy, intracellular, or secreted short proteins with commendable accuracy. The decision to train and tune a feedforward neural net was in part motivated by future adaptability and evaluation scores. For example, in future iterations, data that requires an intermediate representation might be necessary, and therefore neural nets are particularly advantageous for this task. Moreover, following training and testing of this neural net, it performed quite well with a robust 0.95 F1 score for the random/decoy prediction class. Because two distinct “intracellular” and “secreted” clusters in reduced dimensional space are visually discernable, the neural net is trained to classify these two subclasses of positively treated microproteins, with F1 scores of 0.73 for intracellular predictions and 0.68 for secreted classes. While these are commendable performance scores, users should exert caution when examining intracellular vs. secreted predictions, and there is room for improvement to further classify these two distinct classes of microproteins in future iterations.

The versatility of a prediction model of the present system and method is demonstrated by applying the prediction model to various sample types. First, the number of predicted microproteins in a de novo transcriptome that had previously been assembled is counted. These smORFs are annotated by carrying out a three-frame translation step. After running this putative smORF set through the prediction model of the present system and method, each CDS of each smORF is counted, which leads to a total count of 36,025 predicted microproteins. About two-thirds of these predictions were shared between HEK293T and K562 cells.

When visually inspecting many of these predicted microproteins on genome tracks such as UCSC, a distinct pattern emerged: a significant fraction of predicted microproteins were encoded by noncanonical spliced transcript isoforms of known protein-coding genes. Therefore, the types of smORFs predicted are quantified. Indeed, the largest category of predicted microproteins were encoded by transcript isoforms annotated by ENSEMBL as “nondefined CDS.” Nearly a third of total predicted microproteins were encoded by smORFs that exist in these lower abundant ndORFs retained intron transcripts ORFs (riORFs).

Interestingly, recent GENCODE statistics describe that for 19,395 protein-coding genes, there are 89,910 unique transcripts, with 25,082 being partial-length coding. There are an additional 21,427 transcripts labeled as nonsense mediated decay transcripts due to premature stop codons with in the main ORF. In total, the combination of partial-length coding transcripts and nonsense mediated decay transcripts represent over 40% of transcripts that include a segment of protein-coding genes. In a prediction model of the present system and method an additional 3.5% of our predicted microproteins mapping to nonsense mediated decay transcripts (nmdORFs). Altogether, this suggests that a large fraction of predicted microproteins (i.e., the least likely to be random) are encoded by riORFs, ndORFs, and nmdORFs.

Whereas the majority of smORFs called by ribosome profiling workflows are uORFs, using the prediction model of the present system and method just 7.8% of predictions mapping to uORFs. The difference in uORFs can be explained by multiple factors. For example, the active translation of uORFs might primarily function as cis-regulators of the canonical ORFs. Consider the nonsense-mediated decay (NMD) pathway in the example of a translating uORF. The NMD system ensures permissive translation of mRNA following the pioneering round of translation. If mRNA is translated, but the ribosome encounters a premature stop codon (typically defined as 50 base pairs prior to an exon-junction complex), the NMD mechanism is triggered, leading to mRNA degradation (50, 51). A uORF would, therefore, follow a pattern of NMD.

Importantly, some annotated uORFs are not uORFs in the traditional sense, but rather they are simply smORFs in a transcript isoform with exon architecture that likely permits pioneering translation. Examples of this phenomenon include MIEF1-MP and ASDURF1, both of which are smORFs located “upstream” of canonical ORFs at a DNA level (13, 27, 52). However, at the RNA level, they contain unique transcript isoforms that exclude canonical ORF exons, allowing permissive translation of the smORF and the production of a microprotein byproduct (e.g., ENST00000605941.5). For researchers attempting to functionalize microproteins encoded by uORFs, a prediction model of the present system and method is useful because it could highlight the uORFs most likely to encode for functionally active microproteins.

Understanding the microprotein classification of the present system and method is crucial. Preferably, the system and method hereof categorizes microproteins based on their similarity to randomly generated decoy ORFs and provides information about their intracellular or secreted localization. Predictions from some implementations of the present system and method do not imply full integration into the cellular system or high abundance. In fact, considering that most predicted microproteins are encoded by riORFs, ndORFs, and nmdORFs, this implies that predicted microproteins from a transcriptome-defined search space are likely to not be abundant. Instead, the present system and method preferably highlights smORFs that have potential to encode microproteins that could, in certain contexts, be integrated in the cell system.

Recent studies highlight the growing interest in noncanonical proteins, particularly in cancer biology (3, 16, 17, 53). The present system and method becomes particularly pertinent in this context. For example, the study described utilizes the present system and method to investigate the role of predicted microproteins in lung tumors. Using one embodiment of the present system and method nearly a thousand differentially expressed microproteins with large fold changes (absolute value exceeding two) between tumors and normal adjacent tissue, with a significant portion upregulated over 2-fold. Among these, a novel microprotein can be identified encoded by a transcript isoform of TXNDC17, which is currently annotated as lacking a defined CDS by ENSEMBL (i.e., ndORF). This microprotein, predicted by the present system and method was dramatically upregulated in tumors.

In cancer research, de novo proteins have emerged as promising candidates for immunotherapy. These de novo proteins have been engineered based on existing proteins as templates. For example, mimetics based on IL-2 and IL-15 have demonstrated notable immunotherapeutic activity in murine models (54). When discussing microproteins, the term de novo may also refer to natural proteins encoded by alternate ORFs on existing transcripts or, in rarer cases, by new ‘intergenic-like’ transcripts that lack evolutionary conservation. These ORFs may exhibit translational differences due to mutations or mechanisms evading processes such as the NMD pathway, thereby promoting cancer cell proliferation (55, 56). Lindenboom et al. showed that immunoreactivity in tumors with frameshifting mutations evading NMD implies a potential heightened responsiveness to immunotherapy (56).

As an example, employing the present system and method to experimentally target a novel microprotein encoded by an uoORF on the StAR transcript, termed StAR-MP. This microprotein spans 109 amino acids and features an 11-residue-long poly-alanine region that would be challenging to map via Ribo-Seq. The active translation of StAR-MP has not been found by Ribo-Seq methods. One form of the present system and method initially classified StAR-MP into the ‘intracellular’ class with a confidence level of 99%. Simultaneously, StAR-MP was detected in K562 cell lysates using MS. The canonical StAR ORF encodes a protein pivotal in cholesterol import into the mitochondria, serving as a regulatory factor in the steroid synthesis pathway. Notably, the StAR transcript is abundantly enriched in cells of tissues responsible for steroid production.

To further characterize StAR-MP, a specific antiserum against StAR-MP was produced and validated its specificity using negative HEK293T cells and HEK293T cells expressing a StAR-MP construct. Importantly, using this StAR-MP antiserum, showed endogenous expression of this microprotein in normal human testis and ovary tissue extracts. A common approach in microprotein functional experiments is to add a fusion tag to microprotein sequences to identify possible binding partners using immunoprecipitation and to study intracellular localization. However, tagging microproteins with charged fusion tags (e.g. FLAG tag) or much larger proteins (e.g. Fc domain or GFP) can interfere with their native function and localization. Experimentally, not tagging StAR-MP, and instead using specific antiserum to infer stability through western blot and immunofluorescence experiments. Additionally, using a custom RIA, we detected StAR-MP in a dose-dependent manner in steroid-producing tissues where StAR protein transcript is highly expressed. Notably, although StAR protein is more abundant in adrenals relative to gonads, the opposite case is found for the StAR uoORF microprotein, albeit a limited number of human samples were analyzed. Importantly, StAR-MP was not detected in tissues and cells lacking the StAR transcript. Overall, these findings underscore the utility of the present system and method—from prediction and MS identification to experimental functionalization of a microprotein.

Utilization of the present system and method in microprotein discovery is useful, but some precautions offer insights on its optimal use. For researchers seeking to characterize smORFs as potential novel microprotein encoders from an extensive list, the present system and method can serve as an additional filtering tool. For instance, researchers can employ the present system and method to filter Ribo-Seq hits, identifying smORFs that deviate the most from a random ORF. This approach enables prioritization for further investigation into the potential bona fide coding function of selected smORFs.

Moreover, the present system and method proves beneficial for researchers facing limitations in accessing sample types suitable for Ribo-Seq or proteogenomic studies. It provides the ability to assign probabilities of being random, intracellular, or secreted classes based solely on genomic coordinates (e.g., GTF file). This information can be leveraged to generate targeted libraries or peptides for subsequent functional experiments. However, caution is advised as most RNA-Seq data is derived from short-read sequencing, posing challenges in accurately distinguishing a smORF coding sequence as a riORF vs. a uORF. In cases where a microprotein is predicted but the transcript architecture in the sample type does not support active translation, users should exercise caution. To address this, reducing 36,025 predictions to approximately ˜2,000 by considering predictions with over 100 RNA counts (from RNA-Seq) yields a probability assignment exceeding 90%. As long-read sequencing becomes more prevalent, new features can be incorporated into predictive algorithms to better discern these smORFs. Therefore, when applying the present system and method hereof to de novo transcriptomes, subsequent functional experimentation such as CRISPR screens should be used to validate findings.

In general, given the features used for the present system and method, the prediction should be interpreted as “least like a random ORF,” rather than “a new short protein that is integrated into a resting cell system.” Overall, the present system and method represents an innovative approach to microprotein discovery, distinguished by its unique random/decoy computational step. Given the nascent stage of the microprotein field, the present system and method is a valuable addition to current discovery approaches, with the potential to accelerate and refine the field.

### Exemplary Methods

### Datasets Used for Training

In one implementation, two types of datasets for training were used. The first dataset consists of actively translated smORFs, where each the exonic coordinates were downloaded from the Mudge et al., 2022 report (otherwise known as the GENCODE smORF Phase 1 catalog) and merged with the ENSEMBL canonical transcript coordinates to generate a GTF file. The second dataset type consists of short proteins entry IDs from UniProt/Swiss-Prot Reviewed with associated Gene Ontology (cellular compartment) terms and ENSEMBL IDs. The coordinates of these protein-coding genes were downloaded from GENCODE v43 in GTF format. The GENCODE smORFs were given a “ToBePredicted” associated term; this term served as an unknown label. Only ORFs that encode proteins between 30-150 amino acids were selected.

For both dataset types, the DNA and amino acid sequences of each ORF were extracted from their respective GTF files using custom scripts in python. A user should be aware of the order in which the CDS is listed in the GTF file for negative-sensed ORFs. For each ORF, the amino acid sequence and nucleotide sequences were extracted from the 5′ upstream and 3′ downstream genomic location (specified length by the user; e.g., 25 nucleotides) and CDS.

Following sequence extraction, a random/decoy dataset was generated matched to the first dataset type of “functionally unknown” smORFs from the GENCODE smORF Phase 1 catalog. Matching was done to ensure the distribution of protein length was equal to the GENCODE smORFs with unknown function by specifically generating a set of ORFs by mean, 1.25 standard deviations of the distribution, and nucleotide frequency of the upstream and downstream region.

With a random/decoy set generated, three classes of data were then used for training: GENCODE smORFs (with an unknown classification label), UniProt/Swiss-Prot Reviewed (with a positive classification label, sub-classified as intracellular or secreted based on GO cellular compartment term), and random/decoy (with a negative classification label). The generation of a true negative label via random/decoy generation serves as the distinct advantage of the present training model.

### ORF Sequence Feature Representation

In some embodiments, the present system and method represents each ORF by extracting both nucleotide and amino sequence features. For nucleotide features, a user specific k-mer (e.g., 4) for the 5′ and 3′ region is extracted, as well as the first 50 nucleotides of the CDS, chosen based on previous reports on RNA translation efficiency. In addition, a singular kozak score is extracted.

For protein features, four categories of representations were extracted: Ctd, Cksaap, Apaac, and Qso. These were extracted using the protelearn package in Python. Ctd categorizes amino acids into seven classes based on dipole and side chain volumes, originally designed for predicting protein-protein interactions. Cksaap represents a 2-spaced amino acid pair composition of each sequence. Apaac captures information about the sequence order and distribution of hydrophobic amino acids, useful for predicting protein fragment antigenicity and globular protein conformation. Qso captures information about the spacing between amino acids and has been applied in predicting protein subcellular location. These features were carefully selected to reflect the latest understanding of microprotein biochemistry. However, the present system and method may include flexibility so that users can input their own features to train their own models.

### Label Propagation

Following feature extraction, UMAP was used to reduce the dimensionality of the feature space to three dimensions. Then, a LabelSpreading algorithm with a radial basis function (RBF) kernel was used. The algorithm iteratively tunes the gamma parameter until a suitable ratio of between-group to within-group variance was achieved. In this case, the groups include random/decoy, intracellular, and secreted. Therefore, each ORF will be assigned one of these three labels, effectively treating the GENCODE smORFs as unlabeled. As a result, the GENCODE smORF labels are updated to maximize information for downstream machine learning model training. This iterative approach aims to enhance the separation of classes in the label space. The resulting label-propagated data is visualized using a 3D scatter plot generated, offering insight into the distribution of propagated labels in the reduced three-dimensional space. If gamma exceeds 100, a warning is printed to the user to indicate that class separation doesn't appear and thus label propagation may not be recommended. Users may opt to skip label propagation. The primary purpose of UMAP is visual representation and may not represent the higher dimensional features accurately.

### Neural Net Training and Testing

Expanding in more detail on FIG. 6 and accompanying text above, a neural network in one implementation may include an initial input layer, covering both DNA and amino acid features in our case, and an output layer with three nodes for classification (e.g., random/decoy, intracellular, and secreted). Employing the Keras Functional API, the model incorporates crucial components such as batch normalization, activation functions (ReLU), dropout layers, and dense layers. The dataset is divided into training and testing sets with an 80:20 ratio, and hyperparameters are fine-tuned through a randomized search with cross-validation. The following parameters can be fine-tuned: batch size, epochs, validation split, layer one nodes, addition of layer two and number of nodes, dropout rate, and learning rate. The tuning is tone in grid format with cross validation of 3. To obtain the prediction score for each of the specified classes (e.g., random/decoy, intracellular, and secreted), input to a dense layer with a SoftMax activation is used.

Precision, recall, and F1-score metrics are computed to provide a nuanced perspective during testing per class. Precision, representing the accuracy of positive predictions, and recall, indicating the proportion of actual positives correctly predicted, contribute to a comprehensive understanding of the model's balance between false positives and false negatives for the random/decoy, intracellular, and secreted classes. The F1-score, a mean of precision and recall, is particularly informative when seeking a balance between these metrics.

### Cell Culture

Three cell lines were used to generate our de novo transcriptome, which has been previously reported by Martinez et al., 2020). HEK293T cells (HCL4517) were purchased from GE Life Sciences (Pittsburgh, PA). K562 cells (89121407) were purchased from MilliporeSigma (Carlsbad, CA). HEK293T cells were maintained in DMEM (10-013-CV, Corning, San Diego, CA) supplemented with 10% Fetal Bovine Serum (FBS; Corning, 35-010-CV). K562 cells were maintained in RPMI 1640 (Corning, 10-040-CV) supplemented with 10% FBS. All cells were maintained at 37° C. with 5% CO2.

### Applying to a De Novo Transcriptome

Using known transcriptomic data, both poly-A and non-poly-A RNA were captured and sequenced with 250 M reads for each cell line. Reads were aligned using STAR with the following commands: --outSAMstrandField intronMotif --sjdbOverhang 149 --sjdbScore 2 --outFilterMultimapNmax 2 --outFilterScoreMinOverLread 0.25 --outFilterMatchNminOverLread 0.25 --outFilterMismatchNoverReadLmax 0.04 --alignIntronMin 20 --alignIntronMax 1000000 --alignMatesGapMax 1000000 -alignSJoverhangMin 12 --alignSJDBoverhangMin 1. From there, StringTie was used to assemble transcripts with guided assembly option based on the GENCODE reference transcriptome, as previously described. These assembled transcripts were then fed through a custom program called GTFtoFASTA to in silico translate smORFs (11). Briefly, ORFs were annotated by identifying the most distal in-frame upstream ATG start codon for every stop codon across all three reading frames.

In total, 1,192,422 putative smORF queries extracted from this HEK293T/K562 transcriptome that have potential to encode microproteins between 30-150 amino acids in length with ATG start codons. These smORFs were annotated in GTF format and patched together to their native transcript as annotated by StringTie using a custom python script. The resulting GTF file that contained the smORF CDS and native transcript were then fed through the prediction mode pipeline, which then extracted all sequences and features, scaled the data appropriately based on the original scaling parameters during training, and then were assigned a probability per class. After performing MS on HEK293T and K562 cells, each CDS of each predicted microprotein is counted using the DESeq package in R, which borrows from HTSeq (57).

Annotating Microproteins Based on its smORF Type, Evolutionary Conservation, and Sequence Similarity

Each predicted microprotein was the assigned a smORF type based on its genomic location. For example, if a smORF lies in the 5′ UTR of an annotated, canonical protein-coding gene, then the predicted microprotein is described as a uORF. Each smORF was annotated based on the intersect method from the BEDTools suite, which identifies overlapping regions between smORFs and the ENSEMBL reference annotation. Since a smORF may overlap multiple transcript types (e.g., the 5′ UTR region and a separate transcript that contains a retained intron), the smORF type is labeled according to the priority as followed: ‘uoORF’, ‘ndORF,’ ‘lncRNA’, ‘uORF’, ‘riORF’, ‘nmdORF’, ‘aiORF’, ‘psORF’, ‘dORF, ‘eORF’, ‘oCDS.’

For evolutionary conservation, analysis, PhyloCSF scores are smoothed for the 29-mammals alignment, extracted from the UCSC genome browser's PhyloCSF Track Hub with the bedtools map function (31). For BLAST analysis, BLASTP is used to compare predicted microproteins against all annotated proteins on GRCh38. Hits with a BLASTP score greater than or equal to 80 are filtered out, focusing on highly similar proteins. This way, a predicted microprotein set is unique and not duplicate entries from canonical or isoform proteins currently annotated. Multiple sequence alignment for the ndORF in FIG. 1F was carried out using Clustal Omega (58).

### Hydrophobicity Calculations

Custom functions are generated to score the hydrophobicity of each microprotein's C-terminus. Written in R, the script utilizes the Peptides library to compute the average hydrophobicity per position within a data frame of amino acid sequences. It employs a specified hydrophobicity scale (‘Miyazawa’) and iterates through sequences, updating a vector with hydrophobicity values for the last 30 amino acids. After processing all sequences, it calculates the average hydrophobicity per position.

### Differential Expression Analysis

Publicly available data from the Sherlock-Lung cohort, which is a study designed to track lung cancer etiology in never smokers, can be analyzed (See e.g. FIG. 11 and accompanying text above). RNA-Seq data in the form of raw fastq files (GSE171415) are downloaded and re-aligned to Hg38 using STAR, using our custom microprotein GTF file that contains splicing junctures through exon annotation. The command line parameters have been set with specific values for various options, as previously described when generating the reference guided transcriptomes. A total of 29 tumors and 31 normal lung tissues were able to be processed and analyzed. After generation of aligned bam files, differential expression analysis was carried out using R package DESeq2. Prior to carrying out the differential analysis, predicted microproteins were counted from the aligned bam files using the summarizeOverlaps function in R, which is derived from the methods found in the HTSeq package. A genomic range object is used and counted predicted microproteins by using the “Union” mode. In this case, due to overlapping genomic features, the inter.feature parameter is set as false. An experimental model examined predicted microprotein expression differences between tumor and normal tissue with batch correction. Principal component analysis was used to reduce the dimensionality of the data. To visualize PCA, the removeBatchEffect function was used from the lima package. To visualize significantly different smORFs, volcano plots were generated using the EnhancedVolcano R package. The overall design of the expression analysis was as followed: design=tissue type+batch.

### Mass Spectrometry Experimental Workflow

MS experiments were carried out using HEK293T cell lysates and K562 cell lysates, with distinct enrichment steps to increase the relative abundance of lower molecular weight proteins. For HEK293T cells, protein lysates were prepared through acid extraction (1 N acetic acid/0.1 N HCl, 50 mM HCl, 0.1% β-mercaptoethanol (β-ME); 0.05% Triton X-100) and subsequently subjected to a Bond Elute C8 solid-phase extraction (Agilent Technologies, Santa Clara, CA). Approximately 100 mg of sorbent was used for 10 mg of total lysate protein. Cartridges were prepared with one column volume of methanol and were equilibrated with two column volumes of triethylammonium formate (TEAF) buffer at a pH of 3.0 before the sample was applied onto them. The resulting eluate in acidified 75% acetonitrile was subjected to lyophilization, followed by resuspension in 1× tris-acetate-SDS buffer. The sample was then treated with 50 mM DTT for reduction and denatured at 50° C. for 10 minutes. The next step involved fractionation on a 12% GELFrEE cartridge in HEPES running buffer (Expedeon). To facilitate analysis, we fractionated 350 μg of total protein into 12 fractions, with each fraction containing 150 μl. Notably, most of the protein, approximately 300 μg out of the total 350 μg, was concentrated in Fraction 1. Consequently, the remaining fractions were distributed with an estimated content of around 5 μg each, considering the significant protein concentration in Fraction 1. Samples were further enriched using high pH reversed phase fractionation as described by the manufacturer (Thermo, #84868). For K562, proteins were extracted by boiling in water followed by sonication and C8 extraction as previously mentioned (6). Then, Superdex 75 size exclusion chromatography columns were used to create 12 distinct fractions prior to MS.

The analysis was conducted with an Orbitrap Fusion Lumos Tribrid mass spectrometer (Thermo Fisher Scientific), where the sample was directly injected into a 25 cm by 100 μm ID column, meticulously packed with BEH 1.7 μm C18 resin (Waters Corporation). This process utilized an Easy nLC 1000 system (Thermo Fisher Scientific) to separate the samples at a precise flow rate of 400 nl/min. The separation buffers consisted of Buffer A (0.1% formic acid in water) and Buffer B (0.1% formic acid in 90% acetonitrile), facilitating a meticulously controlled gradient from 1% to 25% Buffer B over 100 minutes, then elevating to 40% over the next 20 minutes, and further to 90% within an additional 10 minutes, maintaining at 90% Buffer B for the concluding 10 minutes, totaling a 140-minute runtime. Prior to sample injection, the column was equilibrated with 15 μl of Buffer A. Peptides were then eluted directly from the column's tip and nanosprayed into the mass spectrometer, achieved by applying a 2.5 kV voltage at the column's rear. Operating in data-dependent mode, the Lumos mass spectrometer conducted full MS scans within the Orbitrap at a 120K resolution, covering a mass range from 400 to 1500 m/z, with an AGC target of 4e5. The cycle time was optimized to 3 seconds, within which the most abundant ions per scan were selected for CID MS/MS in the ion trap, setting an AGC target of 2e4 and a minimum intensity threshold of 5000. Fill times were capped at 50 ms for MS and 35 ms for MS/MS scans, respectively. The system utilized quadrupole isolation at 1.6 m/z, enabled monoisotopic precursor selection, and applied dynamic exclusion with a 5-second exclusion duration to ensure precise and efficient peptide identification.

For data analysis, MSFragger (v3.8) was used to map the fragmentation of spectra to ShortStop predicted microproteins (59), the human reference proteome, and common contaminants. The experimental parameters were configured with a precursor mass tolerance of 20 ppm and a fragment mass tolerance of 50 ppm. The data acquisition was performed using a data-dependent acquisition approach, and a fragment mass tolerance of 0.02 Da was applied. Additionally, the inclusion of carbamidomethylation (+57.021464 Da) was specified as a fixed modification. As part of the FragPipe pipeline, Percolator was used to calculate False Discovery Rate (FDR).

### Immunopeptidomes Data Analysis

Raw MS data from PXD013649 were downloaded from the PRIDE, consisting of immunoaffinity purified HLA peptides from HB95 cells, HB145 cells, lung tissue, and lung tumos. Raw files were converted to mzML files using ThermoRawFileParser. The mzML files were then used to search for predicted microproteins using the fragpipe pipeline with a workflow specifically designed to capture 7-25 peptide fragments in HLA-1 complexes with an FDR<0.05. A search database included predicted microproteins, the reference human proteome, and common contaminants with reverse appended sequences for a reverse-decoy approach.

### StAR-MP Antiserum

All animal procedures received approval from the Institutional Animal Care and Use Committee of the Salk Institute and adhered to the PHS Policy on Humane Care and Use of Laboratory Animals (PHS Policy, 2015), the U.S. Government Principles for Utilization and Care of Vertebrate Animals Used in Testing, Research, and Training, the NRC Guide for Care and Use of Laboratory Animals (8th edition), and the USDA Animal Welfare Act and Regulations. Animals were housed in an AAALAC accredited facility in a climate controlled environment under 12 hr light/12 hr dark cycles. Rabbits were provided ad libitum feed (5326 Lab Diet, high fiber), micro-filtered water, and weekly fruits and vegetables and alfafa hay for enrichment. Animals were monitored daily by the veterinary staff for good health. Three female New Zealand white rabbits aged 12 to 14 weeks, weighing 3.0 to 3.2 kg at the study's commencement, and sourced from Irish Farms (I.F.P.S. Inc., Norco, California, USA), were used for StAR-MP antisera production.

A synthetic peptide fragment encoding human StAR (4-38), HSLQRGTFKTQNTRSRLQLRDSEAKLEGLRKDEEC, was coupled to keyhole limpet hemocyanin (KLH) via maleimide, following manufacturer's instructions (ThermoFisher, Waltham MA). The peptide was synthesized, C18 HPLC purified to 98%, and amino acid sequence verified by MS by RS Synthesis (Louisville, KY). The immunogen was prepared by emulsification of Freund's complete adjuvant-modified Mycobacterium butyricum (EMD Millipore) with an equal volume of phosphate buffered saline containing 1.0 mg conjugate/ml for the first two injections. For booster injections, incomplete Freund's adjuvant was mixed with an equal volume of PBS containing 0.5 mg conjugate/ml. For each immunization, an animal received a total of 1 ml of emulsion in 20 intradermal sites in the lumbar region, 0.5 mg total protein conjugate for the first two injections and 0.25 mg total protein conjugate for all subsequent booster injections. Three individual rabbits were injected every three weeks and were bled one week following booster injections, <10% total blood volume. Rabbits were administered 1-2 mg/kg Acepromazine IM prior to injections of antigen or blood withdrawal. At the termination of study, rabbits were exsanguinated under anesthesia (ketamine 50 mg/kg and aceprozamine 1 mg/kg, IM) and euthanized with an overdose of pentobarbital sodium and phenytoin sodium (1 ml/4.5 kg of body weight IC to effect). After blood was collected the death of animals was confirmed. All animal procedures were conducted by experienced veterinary technicians, under the supervision of Salk Instute veterinarians.

Each bleed from each animal was tested at multiple doses for the ability to recognize the synthetic peptide antigen. Bleeds exhibiting the highest titers underwent further analysis through western immunoblot to confirm their ability to recognize the recombinantly expressed full-length StAR microprotein. Additionally, they were tested for the ability to detect endogenous StAR microprotein in tissue extracts by competitive radioimmunoassay using 125I-labeled StAR MP synthetic peptide. The StAR antiserum derived from the rabbit (code PBL #7396, 5/05/96 bleed) displaying optimal characteristics in terms of titer against the synthetic peptide antigen and the ability to recognize the endogenous protein was utilized in all experiments.

### StAR-MP Immunofluorescence and Western Blot

StAR-MP cDNA in a pcDNA3.1 construct was overexpressed using HEK293T cells using lipofectamine 2000. Following 24 hours, cells were either fixed with 2% PFA or lysed for western blot analysis. For immunofluorescence, cells were seeded on a ibidi 8-well slides and fixed for 10 minutes in 2% PFA and permeabilized using 0.1% TX-100 for 10 minutes, followed by blocking with 2% BSA for 60 minutes and overnight 4 C antiserum incubation in 2% BSA at a 1:2000 dilution. Before each sequential step, cells were washed three times in PBS at room temperature. The following day, cells were washed with PBS and incubated with goat anti-Rabbit IgG cross-asorbed, Alex Flour 488 (A-11034) for 45 minutes at room temperature. Following secondary antibody incubation, cells were washed and mounted using ibidi Mounting Medium with DAPI and imaged using confocal microscopy. Samples were imaged on an inverted confocal microscope (Zeiss LSM 880 with AiryScan) using a 63×1.4NA oil-immersion objective using ZEN software.

For western blot, following 24 hours transfection, cells were washed with PBS and immediately lysed (50 mM Tris pH 7.6, 150 mM NaCl, 0.5% NP40, 5 mM EDTA, 1 mM sodium fluoride, 0.1 mM sodium orthovanadate, 1 mM DTT, 1×HALT protease inhibitor with EDTA) and cleared via centrifugation. The protein concentration was determined BioRad protein assay (BioRad Laboratories, Hercules CA). Samples were then diluted into 5% 2-mercaptoethanol in LDS buffer and heated at 95° C. for 5 minutes. After reduction, 20 ul of samples at 25 μg or 50 ug were loaded in one individual well of a 12% Bolt Bis-Tris gel (Thermo Fisher). Electrophoresis was carried out for 45 minutes at 165 V. After electrophoresis, gels were cut and placed in 20% ethanol for 5 minutes to prepare for PVDF membrane transfer. Using the iBlot apparatus, proteins from the gel were transferred at 20V for 12 minutes. The gel was saved and Coomassie stained to assess transfer efficiency and protein abundance. The PVDF membrane with proteins that were transferred were blocked in 5% nonfat dry milk (BLOTTO grade, BioRad, Hercules CA) and incubated with StAR-MP antiserum (1:2000 final) in 0.1% Tween-20/Tris buffered saline (TBS-T) containing 5% nonfat dry milk overnight at 4° C. The next day, the membrane was washed four times with TBS-T for 10 minutes each time and then incubated for 60 minutes with goat anti-rabbit IR Dye 800CW (Licor). The membrane was washed four times in TBS-T and then once in TBS, 10 min per wash. Membranes were imaged on the Licor, with standards in the 700 nm channel and ligand bound to primary antibody labeled with secondary antibody in the 800 nm channel.

### Radioimmunoassay

The peptide analog (Tyr38) human StAR (4-38), HSLQRGTFKTQNTRSRLQLRDSEAKLEGLRKDEEY (SEQ ID NO:______), was synthesized, HPLC purified to >98%, and sequence verified by RS Synthesis (Louisville, KY). The peptide was radiolabeled with 125I using chloramine T and purified using a diphenyl HPLC column with a 0.1% trifluoroacetic acid-acetonitrile solvent system for use as a tracer in the RIA. Briefly, rabbit PBL serum was used at a final dilution of 1/60,000, and synthetic (Tyr38) human StAR(4-38) was used as a standard. Human tissues, obtained from the Cooperative Human Tissue Network, were extracted in 1 N acetic acid/0.1 N HCl, centrifuged, and supernatant enriched for microproteins using C8 silica cartridges as described (6). Cells in culture, endogenously expressing StAR microprotein or transfected with StAR DNA constructs, were extracted, and purified as described (6). Lyophilized tissue or cell extracts were reconstituted in RIA assay buffer, pH checked and adjusted if necessary, and three to seven dose levels were tested. Free tracer was separated from tracer bound to the antibody with the addition of sheep anti-rabbit g-globulins and 10% (wt/vol) polyethylene glycol. The minimum detectable dose and ED50 for (Tyr38) human StAR(4-38) peptide ranged from 1-2 and 15-20 μg/tube, respectively. Results were calculated using a logit/log RIA data processing program developed by Faden, Hutson, Munson, and Rodbard (NICHD, NIH).

The present disclosure includes references to “embodiments,” which are non-limiting implementations of the disclosed concepts. References to “an embodiment,” “one embodiment,” “a particular embodiment,” “some embodiments,” “various embodiments,” and the like do not necessarily refer to the same embodiment. A large number of possible embodiments are contemplated, including specific embodiments described in detail, as well as modifications or alternatives that fall within the spirit or scope of the disclosure. Not all embodiments will necessarily manifest any or all of the potential advantages described herein.

This disclosure may discuss potential advantages that may arise from the disclosed embodiments. Not all implementations of these embodiments will necessarily manifest any or all of the potential advantages. Whether an advantage is realized for a particular implementation depends on many factors, some of which are outside the scope of this disclosure. In fact, there are a number of reasons why an implementation that falls within the scope of the claims might not exhibit some or all of any disclosed advantages. For example, a particular implementation might include other circuitry outside the scope of the disclosure that, in conjunction with one of the disclosed embodiments, negates or diminishes one or more the disclosed advantages. Furthermore, suboptimal design execution of a particular implementation (e.g., implementation techniques or tools) could also negate or diminish disclosed advantages. Even assuming a skilled implementation, realization of advantages may still depend upon other factors such as the environmental circumstances in which the implementation is deployed. For example, inputs supplied to a particular implementation may prevent one or more problems addressed in this disclosure from arising on a particular occasion, with the result that the benefit of its solution may not be realized. Given the existence of possible factors external to this disclosure, it is expressly intended that any potential advantages described herein are not to be construed as claim limitations that must be met to demonstrate infringement. Rather, identification of such potential advantages is intended to illustrate the type(s) of improvement available to designers having the benefit of this disclosure. That such advantages are described permissively (e.g., stating that a particular advantage “may arise”) is not intended to convey doubt about whether such advantages can in fact be realized, but rather to recognize the technical reality that realization of such advantages often depends on additional factors.

Unless stated otherwise, embodiments are non-limiting. That is, the disclosed embodiments are not intended to limit the scope of claims that are drafted based on this disclosure, even where only a single example is described with respect to a particular feature. The disclosed embodiments are intended to be illustrative rather than restrictive, absent any statements in the disclosure to the contrary. The application is thus intended to permit claims covering disclosed embodiments, as well as such alternatives, modifications, and equivalents that would be apparent to a person skilled in the art having the benefit of this disclosure.

For example, features in this application may be combined in any suitable manner. Accordingly, new claims may be formulated during prosecution of this application (or an application claiming priority thereto) to any such combination of features. In particular, with reference to the appended claims, features from dependent claims may be combined with those of other dependent claims where appropriate, including claims that depend from other independent claims. Similarly, features from respective independent claims may be combined where appropriate.

Accordingly, while the appended dependent claims may be drafted such that each depends on a single other claim, additional dependencies are also contemplated. Any combinations of features in the dependent that are consistent with this disclosure are contemplated and may be claimed in this or another application. In short, combinations are not limited to those specifically enumerated in the appended claims.

Where appropriate, it is also contemplated that claims drafted in one format or statutory type (e.g., apparatus) are intended to support corresponding claims of another format or statutory type (e.g., method).

Because this disclosure is a legal document, various terms and phrases may be subject to administrative and judicial interpretation. Public notice is hereby given that the following paragraphs, as well as definitions provided throughout the disclosure, are to be used in determining how to interpret claims that are drafted based on this disclosure.

References to a singular form of an item (i.e., a noun or noun phrase preceded by “a,” “an,” or “the”) are, unless context clearly dictates otherwise, intended to mean “one or more.” Reference to “an item” in a claim thus does not, without accompanying context, preclude additional instances of the item. A “plurality” of items refers to a set of two or more of the items.

The word “may” be used herein in a permissive sense (i.e., having the potential to, being able to) and not in a mandatory sense (i.e., must).

The terms “comprising” and “including,” and forms thereof, are open-ended and mean “including, but not limited to.”

When the term “or” is used in this disclosure with respect to a list of options, it will generally be understood to be used in the inclusive sense unless the context provides otherwise. Thus, a recitation of “x or y” is equivalent to “x or y, or both,” and thus covers 1) x but not y, 2) y but not x, and 3) both x and y. On the other hand, a phrase such as “either x or y, but not both” makes clear that “or” is being used in the exclusive sense.

A recitation of “w, x, y, or z, or any combination thereof” or “at least one of . . . w, x, y, and z” is intended to cover all possibilities involving a single element up to the total number of elements in the set. For example, given the set [w, x, y, z], these phrasings cover any single element of the set (e.g., w but not x, y, or z), any two elements (e.g., w and x, but not y or z), any three elements (e.g., w, x, and y, but not z), and all four elements. The phrase “at least one of . . . w, x, y, and z” thus refers to at least one element of the set [w, x, y, z], thereby covering all possible combinations in this list of elements. This phrase is not to be interpreted to require that there is at least one instance of w, at least one instance of x, at least one instance of y, and at least one instance of z.

Various “labels” may precede nouns or noun phrases in this disclosure. Unless context provides otherwise, different labels used for a feature (e.g., “first circuit,” “second circuit,” “particular circuit,” “given circuit,” etc.) refer to different instances of the feature. Additionally, the labels “first,” “second,” and “third” when applied to a feature do not imply any type of ordering (e.g., spatial, temporal, logical, etc.), unless stated otherwise.

The phrase “based on” or is used to describe one or more factors that affect a determination. This term does not foreclose the possibility that additional factors may affect the determination. That is, a determination may be solely based on specified factors or based on the specified factors as well as other, unspecified factors. Consider the phrase “determine A based on B.” This phrase specifies that B is a factor that is used to determine A or that affects the determination of A. This phrase does not foreclose that the determination of A may also be based on some other factor, such as C. This phrase is also intended to cover an embodiment in which A is determined based solely on B. As used herein, the phrase “based on” is synonymous with the phrase “based at least in part on.”

The phrases “in response to” and “responsive to” describe one or more factors that trigger an effect. This phrase does not foreclose the possibility that additional factors may affect or otherwise trigger the effect, either jointly with the specified factors or independent from the specified factors. That is, an effect may be solely in response to those factors, or may be in response to the specified factors as well as other, unspecified factors. Consider the phrase “perform A in response to B.” This phrase specifies that B is a factor that triggers the performance of A, or that triggers a particular result for A. This phrase does not foreclose that performing A may also be in response to some other factor, such as C. This phrase also does not foreclose that performing A may be jointly in response to B and C. This phrase is also intended to cover an embodiment in which A is performed solely in response to B. As used herein, the phrase “responsive to” is synonymous with the phrase “responsive at least in part to.” Similarly, the phrase “in response to” is synonymous with the phrase “at least in part in response to.”

Within this disclosure, different entities (which may variously be referred to as “units,” “circuits,” other components, etc.) may be described or claimed as “configured” to perform one or more tasks or operations. This formulation—[entity]configured to [perform one or more tasks]—is used herein to refer to structure (i.e., something physical). More specifically, this formulation is used to indicate that this structure is arranged to perform the one or more tasks during operation. A structure can be said to be “configured to” perform some tasks even if the structure is not currently being operated. Thus, an entity described or recited as being “configured to” perform some tasks refers to something physical, such as a device, circuit, a system having a processor unit and a memory storing program instructions executable to implement the task, etc. This phrase is not used herein to refer to something intangible.

In some cases, various units/circuits/components may be described herein as performing a set of task or operations. It is understood that those entities are “configured to” perform those tasks/operations, even if not specifically noted.

The term “configured to” is not intended to mean “configurable to.” An unprogrammed FPGA, for example, would not be considered to be “configured to” perform a particular function. This unprogrammed FPGA may be “configurable to” perform that function, however. After appropriate programming, the FPGA may then be said to be “configured to” perform the particular function.

For purposes of United States patent applications based on this disclosure, reciting in a claim that a structure is “configured to” perform one or more tasks is expressly intended not to invoke 35 U.S.C. § 112(f) for that claim element. Should Applicant wish to invoke Section 112(f) during prosecution of a United States patent application based on this disclosure, it will recite claim elements using the “means for” [performing a function] construct.

What is claimed is:

1. A method, comprising:
accessing, by a computer system, a set of data describing expressed amino acid sequences;
generating, by the computer system, decoy training data that includes randomized amino acid sequences that are matched to properties of the amino acid sequences included in the set of data; and
training, by the computer system, a machine learning model using labeled training data and the decoy training data, the labeled training data including amino acid sequences within a set of classifications and the decoy training data constituting examples of amino acid sequences that are not expected to be within the set of classifications;
wherein the trained machine learning model is usable to:
receive an input that describes a structure of a particular amino acid sequence; and
perform a classification of the particular amino acid sequence relative to the set of classifications.

2. The method of claim 1, wherein the set of data includes information describing unlabeled amino acid sequences having unknown classifications.

3. The method of claim 2, wherein the labeled training data includes proteins from Swiss-Prot, and wherein the unlabeled amino acid sequences include Ribo-Seq derived ORFs, including GENCODE small open reading frames (smORFs) having unknown classifications.

4. The method of claim 2, further comprising:
performing feature extraction on amino acid sequences described in the labeled training data and the decoy training data; and
labeling, based on information derived from the feature extraction, the unlabeled amino acid sequences in the set of data to create additional labeled training data;
wherein the machine learning model is also trained using the additional labeled training data.

5. The method of claim 4, wherein extracted features resulting from the feature extraction include nucleotide features and amino acid features.

6. The method of claim 5, wherein the nucleotide features include one or more of 4-mers of the 5′ UTR, 3′ UTR, first 50 CDS.

7. The method of claim 5, wherein the amino acid features include one or more of CTDD, CTD, APAAC, QSO.

8. The method of claim 1, wherein the randomized amino acid sequences included in the decoy training data are matched to lengths and codon probabilities of the expressed amino acid sequences included in the set of data.

9. The method of claim 1, wherein classes in the set of classifications include intracellular, secreted, and negative.

10. The method of claim 1, wherein the trained machine learning model is usable to:
perform the classification of the particular amino acid sequence relative to the set of classifications by generating probabilities that the particular amino acid sequence is in one or more of the set of classifications.

11. The method of claim 1, wherein the particular amino acid sequence includes 150 or fewer amino acids.

12. The method of claim 1, further comprising:
receiving, by the trained machine learning model, a description of the particular amino acid sequence;
performing, by the trained machine learning model, a classification of the particular amino acid sequence relative to the set of classifications.

13. A non-transitory computer-readable medium storing program instructions executable by a computer system to perform operations in a training mode that include:
accessing labeled training data that describes amino acid sequences with known classifications;
generating decoy training data that includes randomized amino acid sequences with properties that are matched to properties of actual amino acid sequences, the decoy training data constituting negative training examples; and
training a machine learning model using the labeled training data, and the decoy training data, the trained machine learning model being usable to classify unknown amino acid sequences into one of a set of classifications.

14. The non-transitory, computer-readable medium of claim 13, wherein the randomized amino acid sequences included in the decoy training data are matched to lengths and codon probabilities of the actual amino acid sequences.

15. The non-transitory, computer-readable medium of claim 14, wherein the actual amino acid sequences include GENCODE small open reading frames (smORFs) having unknown classifications.

16. The non-transitory, computer-readable medium of claim 13, wherein the program instructions are executable by the computer system to perform operations in a prediction mode that include:
receiving an input that describes a structure of a particular amino acid sequence; and
using the trained machine learning model to perform a classification of the particular amino acid sequence into one of a set of classifications.

17. The non-transitory, computer-readable medium of claim 16, wherein the set of classifications are customizable by a user.

18. A system, comprising:
one or more processor circuits;
memory storing program instructions executable by the one or more processor circuits to perform operations including, in a training mode:
accessing a set of data describing expressed amino acid sequences;
generating decoy training data that includes randomized amino acid sequences that are matched to properties of the amino acid sequences included in the set of data; and
training a machine learning model using a labeled training set and the decoy training set, the labeled training set including proteins with known classes and the decoy training set constituting negative training examples, the trained machine learning model being usable to classify unknown proteins.

19. The system of claim 18, wherein the randomized amino acid sequences included in the decoy training data are matched to lengths and codon probabilities of the actual amino acid sequences.

20. The system of claim 18, the operations further including, in a prediction mode:
receiving an input that describes a particular amino acid sequence;
using the trained machine learning model to perform a classification of the particular amino acid sequence into one of a set of classifications.

21. A method, comprising:
receiving, by a computer system, an input that describes an amino acid sequence of unknown classification;
using, by the computer system, a machine learning model to perform a classification of the amino acid sequence into one of a set of classifications;
wherein the machine learning model is trained by a computer-implemented process that includes:
accessing a set of data describing expressed amino acid sequences;
generating decoy training data that includes randomized amino acid sequences that are matched to properties of the amino acid sequences included in the set of data; and
training a machine learning model using labeled training data and the decoy training data, the labeled training data including amino acid sequences within a set of classifications and the decoy training data constituting examples of amino acid sequences that are not expected to be within the set of classifications.

22. The method of claim 21, wherein the randomized amino acid sequences included in the decoy training data are matched to lengths and codon probabilities of the actual amino acid sequences.
